(19) United States
(12) Patent Application Publication
(10) Pub. No.: US 2017/0042439 A1
(43) Pub. Date: Feb. 16, 2017

(54) SYSTEM, DEVICE AND METHODS FOR BRAINWAVE-BASED TECHNOLOGIES

(71) Applicant: NATIONAL UNIVERSITY OF SINGAPORE, Singapore (SG)

(72) Inventors: Chen Hua YEOW, Singapore (SG); Yuchun LIU, Singapore (SG); Shruthi SURESH, Singapore (SG)

(73) Assignee: NATIONAL UNIVERSITY OF SINGAPORE, Singapore (SG)

(21) Appl. No.: 15/118,739

(22) PCT Filed: Feb. 13, 2015
(86) PCT No.: PCT/SG2015/000045

§ 371 Date: Aug. 12, 2016

Related U.S. Application Data
(60) Provisional application No. 61/939,878, filed on Feb. 14, 2014.

(51) Int. Cl. A61B 5/0478; A61B 5/04; G06Q 30/02; A61B 5/16; G06F 3/01; G06F 19/00; A61B 5/00; A61B 5/048

(52) U.S. Cl. CPC A61B 5/0478; A61B 5/0006; A61B 5/04015; A61B 5/048; A61B 5/165; A61B 5/4839; A61B 5/6803; G06F 3/015; G06F 19/322; G06F 19/3418; G06Q 30/0277; A61B 2560/0431; B60W 50/08

(57) ABSTRACT

System, device and methods for brainwave-based technologies. The system for measuring and processing brainwave data of a user comprises one or more electrodes for measuring the brainwave data of the user, and a processing unit coupled to the electrodes and configured to process the brainwave data for determining a current mental state of the user and to generate, based on the current mental state of the user, a control signal for instructing activation of a means for manipulating the current mental state of the user.

### FIELD OF INVENTION

The present invention relates broadly to the field of brainwave-based technologies.

### BACKGROUND

To date, the applications of the technology of electroencephalography (EEG) are limited and confined for use by only a specific group of users and is costly. It is desirable to make this technology readily available to general consumers for use in a wide range of applications that can be applied in daily lives.

Some existing indirect measurement methods of the actual brain emotional states are typically performed through facial emotion recognition techniques, electrical skin activity measurements and voice recognition approaches. However, such methods are only indirect and may thus not adequately represent the user's true brainwave state.

As a result, the utility of brainwave data has been under-utilized as a direct measurement of many physiological markers in the body which cannot be understood using indirect methodologies such as skin conductance for example.

Embodiments of the present invention provide a system, device and methods that seek to address at least one of the above problems.

### SUMMARY

In accordance with a first aspect of the present invention there is provided a system for measuring and processing brainwave data of a user, the system comprising one or more electrodes for measuring the brainwave data of the user, and a processing unit coupled to the electrodes and configured to process the brainwave data for determining a current mental state of the user and to generate, based on the current mental state of the user, a control signal for instructing activation of a means for manipulating the current mental state of the user.

In accordance with a second aspect of the present invention there is provided a method for measuring and processing brainwave data of a user, the method comprising providing one or more electrodes for measuring the brainwave data of the user, processing the brainwave data for determining a current mental state of the user; and generating, based on the current mental state of the user, a control signal for instructing activation of a means for manipulating the current mental state of the user.

In accordance with a third aspect of the present invention there is provided a device for measuring brainwave data of a user, the device comprising a portable instrument; and one or more electrodes disposed on, or for disposal on the portable instrument; wherein the portable instrument and/or the electrodes are configured for providing an adjustable configuration of the one or more electrodes for measurement of the brainwave data.

In accordance with a fourth aspect of the present invention there is provided a method for measuring brainwave data of a user, the device comprising providing a portable instrument; providing one or more electrodes disposed on, or for disposal on the portable instrument; and providing an adjustable configuration of the one or more electrodes for measurement of the brainwave data.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1A) shows a schematic diagram illustrating a system according to an example embodiment.

FIG. 1B) shows a schematic diagram illustrating a system according to an example embodiment.

FIGS. 2A)-C) show schematic diagrams illustrating wearable devices according to example embodiments.

FIGS. 3A)-C) show schematic diagrams illustrating wearable devices according to an example embodiment.

FIG. 4 shows graphs illustrating real time data display according to example embodiments.

FIG. 5 shows graphs illustrating real time data display according to example embodiments.

FIG. 6 shows graphs illustrating real time data updating and retrieval according to example embodiments.

FIG. 7 shows a schematic diagram illustrating an algorithm implemented in a system according to an example embodiment.

FIG. 8 shows a schematic diagram illustrating a system according to an example embodiment.

FIG. 9A) shows a schematic diagram illustrating visualization according to an example embodiment.

FIG. 9B) shows a schematic diagram illustrating visualization according to an example embodiment.

FIG. 10 shows a schematic diagram illustrating upload/download according to an example embodiment.

FIG. 11 shows a schematic diagram illustrating emotional diary entries according to an example embodiment.

FIG. 12 shows a schematic diagram illustrating sharing according to an example embodiment.

FIG. 13 shows a schematic diagram illustrating a web store according to an example embodiment.

FIG. 14A) shows a schematic diagram illustrating a hand-held device according to an example embodiment.

FIG. 14B) shows a schematic diagram illustrating a hand-held device according to an example embodiment.

FIG. 15 shows a schematic diagram illustrating a collapsible device according to an example embodiment.

FIG. 16A)-D) show schematic diagrams illustrating a telescopic device according to an example embodiment.

FIG. 17 shows a schematic diagram illustrating a modular device according to an example embodiment.

FIG. 18A)-C) show a schematic diagrams illustrating wearable devices according to example embodiments.

FIGS. 19 shows a schematic diagram illustrating a wearable device according to an example embodiment.

FIGS. 20 shows a schematic diagram illustrating a wearable device according to an example embodiment.

FIGS. 21A)-B) shows schematic diagrams illustrating a wearable device according to an example embodiment.

FIGS. 21C)-D) shows schematic diagrams illustrating wearable devices according to example embodiments.

FIG. 22 shows a schematic diagram illustrating an active intervention according to an example embodiment.

FIG. 23 shows a schematic diagram illustrating an active intervention according to an example embodiment.

FIG. 24 shows a schematic diagram illustrating an EEG pain response application according to an example embodiment.

FIG. 25 shows a schematic diagram illustrating an EEG pain response application according to an example embodiment.

FIG. 26 shows a schematic diagram illustrating a display system with real time EEG response application according to an example embodiment.

FIG. 27 shows a schematic diagram illustrating an online emotion-sharing application according to an example embodiment.

FIG. 28 shows a schematic diagram illustrating a text messaging emotion-sharing application according to an example embodiment.

FIG. 29 shows a schematic diagram illustrating a social network online emotion-sharing application according to an example embodiment.

FIG. 30 shows a schematic diagram illustrating a mall directory with emotion rating application according to an example embodiment.

FIG. 31 shows a schematic diagram illustrating a customer emotion rating application according to an example embodiment.

FIG. 32 shows a schematic diagram illustrating a movie emotion rating application according to an example embodiment.

FIG. 33 shows a schematic diagram illustrating a customer service with emotion rating application according to an example embodiment.

FIG. 30 shows a schematic diagram illustrating a mall directory with emotion rating application according to an example embodiment.

FIG. 34 shows a schematic diagram illustrating an online photo with emotion tagging application according to an example embodiment.

FIG. 35A) shows a schematic diagram illustrating a wearable device with active intervention means according to an example embodiment.

FIG. 35B) shows a schematic diagram illustrating a wearable device with active intervention means according to an example embodiment.

FIG. 35C) shows a schematic diagram illustrating a wearable device with active intervention means according to an example embodiment.

FIG. 36 shows a schematic diagram illustrating integration of a wearable device with an external active intervention means according to an example embodiment.

FIG. 37 shows a schematic diagram illustrating integration of a wearable device with an external active intervention means according to an example embodiment.

FIG. 38 shows a schematic diagram illustrating operation of a wearable device with active intervention means according to an example embodiment.

FIG. 39A) shows plots illustrating active intervention control based on brainwave data according to an example embodiment.

FIG. 39B) shows plots illustrating active intervention control based on brainwave data according to an example embodiment.

FIG. 40 shows a flow chart illustrating a method for measuring and processing brainwave data of a user, according to an example embodiment.

FIG. 41 shows a flow chart illustrating a method for measuring brainwave data of a user, according to an example embodiment.

### DETAILED DESCRIPTION

Embodiments of the present invention relate to the detection and processing and/or display of brainwaves, and utilizing the brainwave data for a variety of different applications with the aim of improving performance, quality of life and/or healthcare.

A system according to example embodiment comprises of a brainwave-sensing device and associated brainwave detection-interpretation software. Active interventions for manipulating a current mental state of the user can be built-in or external to the device. In different embodiments, the device can be implemented without provision of any interventions. The brainwave-sensing device, a lightweight and portable instrument, will for example be worn on a specific region of the head to track the brainwaves of the user, and is fitted with dry electrodes that provide improved user handling and comfort. The brainwave detection-interpretation device can be incorporated into mechanical structures, such as, but not limited to, furniture (e.g. chairs, sofas, bed/pillows, vehicle seats etc.), windows and walls, and/or can be incorporated with electronic devices, such as, but not limited to, a smartphone, tablet, laptop, desktop computer, phone, camera, or external instrument, which can be installed with the brainwave detection software to which the collected brainwave signals can be transmitted. The software processes the brainwave signals, for example to identify and display the brainwave states upon calibration of the user's basal brainwave states.

The utility of the detected and processed brainwave data can be broadly categorized into:

(1) communication (such as, but not limited to, social purposes),

(2) Health & wellness, including sports, medical-related purposes, and

(3) Products, services and entertainment (not limited to dining, gaming, movies, remote control gadgets/toys etc.)

Some examples of how the collected brainwave data can be used in example embodiments include, but are not limited to, centralized brainwave data compilation system, self-improvement, remote brainwave-detection and brainwave-sharing via digital platforms, brainwave-tagging of various digital media, brainwave-monitoring for social and healthcare reasons, brainwave-based rating of products and services, brainwave-targeted advertisements, brainwave-induced drug release, communication via brainwave-engagement with illness-stricken parties who are in comatose, stroke and/or unable to communicate or express themselves, as well as brainwave-modulated social robots. Further applications can include brainwave-controlled toys and assistive devices for general consumers and physically-disabled, sleep quality management for sports and wellness (including sleep apnea detection-intervention), brainwave-monitoring for both static and dynamic athletic tasks, and brainwave sensor-embedded headrests for drivers and passengers.

In one embodiment, a brainwave-sensing device is provided which allows for the detection and display of mental states such as emotions (happiness, anger, sadness, fear, excitement), pain, anxiety, sleep, mental fatigue, comfort and pleasure. The device can come in various forms and shapes, such as, but not limited to, a wearable device and can be a hand-held device depending on the application. In the description and claims, the term “portable” is used as including, at a minimum, a wearable device and a hand-held device. The form factor of the portable device can be further modified and/or customized in different embodiments to suit individual use-cases and/or personal preferences.

The portable device has at least one, and preferably multiple dry electrodes (and optionally other sensors or sensing devices) which can be configured in multiple ways to allow for different use cases. The software in example embodiments uses supervised/unsupervised algorithm(s) to detect the mental states, e.g. anxiety levels, of the user based on the brainwave data. A supervised approach requires the user to exhibit a specific mental state repeatedly so that the detected brainwave profile can be tagged to the desired mental state. An unsupervised approach uses a predetermined relationship between the specific mental state and the brainwave data, e.g. the brainwave profile, based on previously collected test data from a subject population. The mental state information can be stored on a centralized database for subsequent retrieval or analysis; and/or can be displayed on a computer system or mobile app.

Brainwave states such as, but not limited to, happiness, anger, sadness, pain, anxiety, fear and excitement play an important role in decision-making and planning of daily live. Different parts of the human brain are known to be responsible for specific functions of the human body, as described, for example, in Morris C G, Maisto A A. Psychology: An

Introduction, Eleventh Edition, 2001. For instance, attention is associated with the frontal brain areas, where children with Attention Deficit Hyperactivity Disorder (ADHD) tend to express abnormally high theta activation in the frontal brain areas, as described, for example, in Adam R Clarke, Robert J Barry, Rory McCarthy, Mark Selikowitz, Accepted: Aug. 9, 2001; DOI, http://dx.doi.org/10.1016/S1388-2457(01)00668-X; Brainwave memory is associated with the right brain area near the ear, as described, for example, in Edmonton Neurotherapy Brain Map, http://www.edmontonneurotherapy.com/Edmonton_Neurotherapy_QEEG_brain_mapping.h tm. Erk, S. et al. (2003) Brainwave context modulates subsequent memory effect. Neuroimage 18, 439-447 reported that subjects tend to exhibit activity in the right fusiform gyms and right amygdala when exposed to positive and negative emotional stimuli respectively.

Bekkedal M Y, Rossi J 3rd, Panksepp J. Human brain EEG indices of brainwaves: delineating responses to affective vocalizations by measuring frontal theta event-related synchronization. Neurosci Biobehav Rev. 2011 October; 35(9):1959-70 observed cortical regional differences in electroencephalography (EEG) alpha power shifts during brainwave stimulation, for example, left brain area activation with sad state, right brain area activation with angry state, and left and right brain area activations with happy state. The same study further reported that the greatest theta synchronization exists in the anterior hemisphere and can be gender-specific, such as male subjects responding with substantial theta power to sounds of pleasure and female subjects responding with high theta power to sounds of anger.

Pain and anxiety are known to associate with occipital lobe alpha power and frontal lobe alpha asymmetry respectively, as described, for example, in Nir R R, Sinai A, Raz E, Sprecher E, Yarnitsky D. Pain assessment by continuous EEG: association between subjective perception of tonic pain and peak frequency of alpha oscillations during stimulation and at rest. Brain Res. 2010 Jul 16;1344:77-86 and Briesemeister, B. B., Tamm, S., Heine, A., & Jacobs, A. M. (2013). Approach the good, withdraw from the bad—A review on frontal alpha asymmetry measures in applied psychological research, Psychology, 4(3A), 261-267.

Collectively, studies such as the abovementioned indicate the possibility of associating localized brainwave profiles with various brainwave states. Given an appropriate calibration procedure adapted from the prior literature on affective stimulation, for example Positive and Negative Affect Scale described in Schneider F, Gur R C, Gur RE, Muenz L R. Standardized mood induction with happy and sad facial expressions. Psychiatry Res. 1994 January; 51(1):19-31, International Affective Digitized Sounds described in Bradley, M. M., & Lang, P. J. (1999). International affective digitized sounds (IADS): Stimuli, instruction manual and affective ratings (Tech. Rep. No. B-2). Gainesville, Fla.: The Center for Research in Psychophysiology, University of Florida, or International Affective Picture System described in Lang, P. J., Bradley, M. M., & Cuthbert, B. N. (2005). International affective picture system (IAPS): Affective ratings of pictures and instruction manual. Technical Report A-6, University of Florida, Gainesville, Fla., it is advantageously possible to relate, for each individual subject, different localized brainwave profiles to specific levels of brainwave states.

The present specification also discloses apparatus for implementing or performing the operations of the methods. Such apparatus may be specially constructed for the required purposes, or may comprise a device selectively activated or reconfigured by a computer program stored in the device. Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a device. The computer readable medium may also include a hard-wired medium such as exemplified in the Internet system, or wireless medium such as exemplified in the GSM mobile telephone system. The computer program when loaded and executed on the device effectively results in an apparatus that implements the steps of the method.

The invention may also be implemented as hardware modules. More particular, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the system can also be implemented as a combination of hardware and software modules.

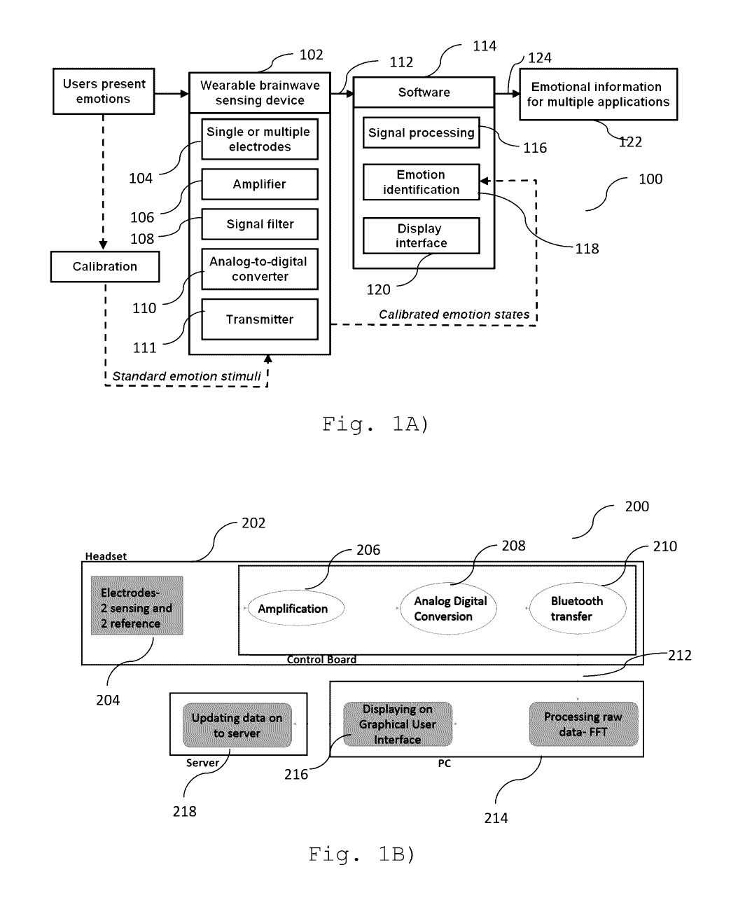

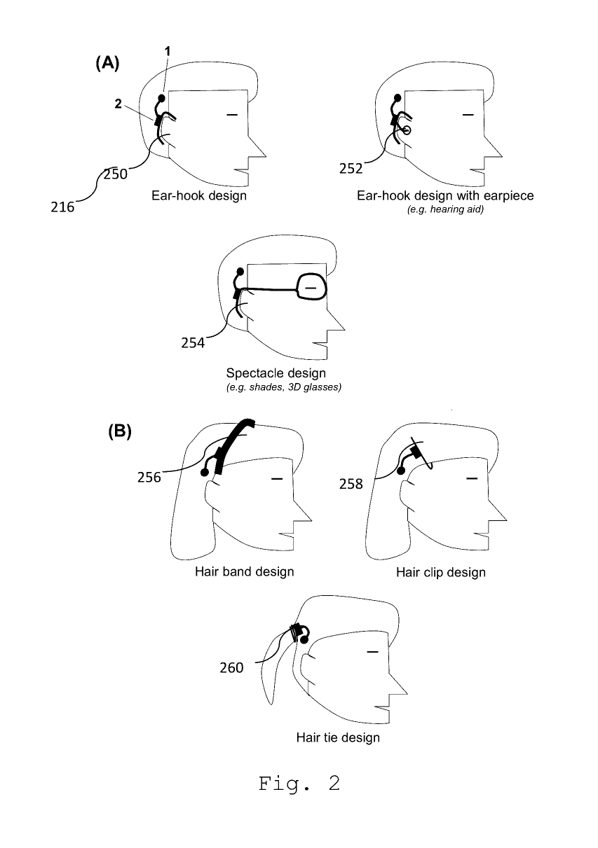

FIG. 1A shows a schematic diagram illustrating a system 100 according to an example embodiment, that comprises a portable instrument such as a wearable brainwave-sensing device 102 that can detect brainwaves via electrodes 104 placed on the scalp, and uses a series of amplifier 106, signal filter 108 and analog-to-digital converter 110 for calibration and subsequent wireless/wired transmission of the detected brainwave signals, indicated at numeral 112, using a transmitter 111. This system 100 and implemented process can be powered by a rechargeable battery or solar cell, which may be integrated in the portable device 102. The system 100 further comprises software 114 that, when running on an appropriate computing device, receives the transmitted brainwave signals 112 and which will subject the signals to signal processing 116 and brainwave interpretation such as emotion identification 118, followed by subsequent display of the brainwave status information on a display interface 120 for viewing. The system 100 also comprises one or more of a wide-ranging scope of different applications 122 in which the identified brainwave information 124 may be used.

By way of non-limiting example only, as electrodes 104 the available component g.Tec Dry g.SAHARAelectrode, 16 mm (https://www.olimex.com/products/eeg/openeeg/eeg-digital-pcb/) may be used, as amplifier 106 and signal filter 108 the available component Olimex EEG-Analog-ASM (https://www.olinex.com/produrts/eeg/openeeg/eeg-analog-pcb/) may be used, as the analog-to-digital converter 110 the available component Olimex EEG-Digital-ASM (https://www.olimex.com/products/eeg/openeeg/eeg-digital-pcb/) may be used, and as the transmitter 111 the available component SparkFun Bluetooth Mate Gold WRL-12580(https://www.sparkfun.com/products/12580) may be used.

FIG. 1B shows a schematic diagram illustrating another system 200 according to an example embodiment, that comprises a portable instrument such as a headset 202 that can detect brainwaves via electrodes 204 (for example two sensing and two reference electrodes) placed on the scalp when the headset 202 is worn by a user, and uses a series of amplifier 206, analog-to-digital converter 208 and a transfer interface, here in the form of a Bluetooth transfer 210 for transmission of the detected brainwave signals, indicated at numeral 212. The system 200 further comprises software running on an appropriate computing device, here a Personal Computer (PC) 214, which receives the transmitted brainwave signals 212 and which will subject the signals to signal processing and brainwave interpretation such as emotion identification, followed by subsequent display of the brainwave status information on a Graphical User Interface (GUI) 216 for viewing. The system 200 in this embodiment also comprises a Server 218 for data storage/accessibility.

A lightweight and portable instrument based on EEG technology can be provided as the brainwave detection device in example embodiments, whereby a single or multiple electrode(s) can be placed on a localized area of the head to detect specific brainwaves of interest of the user. This serves to detect brainwave status information such as, but not limited to, sleep, attention, happiness, anger, sadness, pain, anxiety, fear and excitement, where the electrode placement can be adjusted to suit detection of different brainwave states. For instance, the electrode(s) can be placed near the right ear to detect brainwave states such as joy and anger, or near the forehead to detect attention (Please provide full reference, as it was not in the list of references in the provisional application Clarke et al., 2001; Bekkedal M Y, Rossi J 3rd, Panksepp J. Human brain EEG indices of brainwaves: delineating responses to affective vocalizations by measuring frontal theta event-related synchronization. Neurosci Biobehav Rev. 2011 October; 35(9):1959-70). A reference electrode near the bony areas (e.g. ear bone) is typically used to act as a form of ground signal when amplifying the EEG electrode signal.

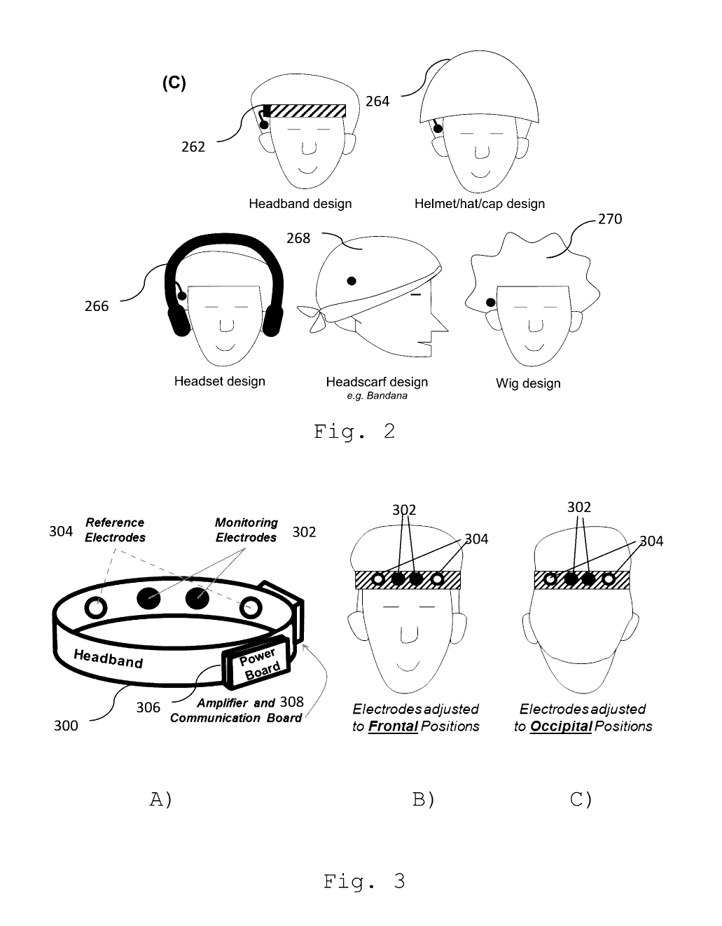

The brainwave detection device may be incorporated into mechanical structures such as, but not limited to, furniture (e.g. chairs, sofas, bed/pillows, vehicle seats etc.), windows and walls. In addition, it can be worn in various ways such as, but not limited to, either as a standalone measurement equipment designed in the form of ear hooks 250, earpiece 252 (such as hearing aids), spectacles 254, hairbands 256, hair clips 258, hair tie 260 (FIGS. 2A) and B)), or headwear/head electronics such as, but not limited to, headband 262, helmet/hat/cap 264, headset 266, wig 268 or head scarf 270 (e.g. bandana), see FIG. 2C), or as add-on accessories to the aforementioned existing items (FIGS. 2A)-C)), each carrying a single or multiple electrodes e.g. 1 and a processing unit e.g. 2 (as labeled in FIG. 1A).

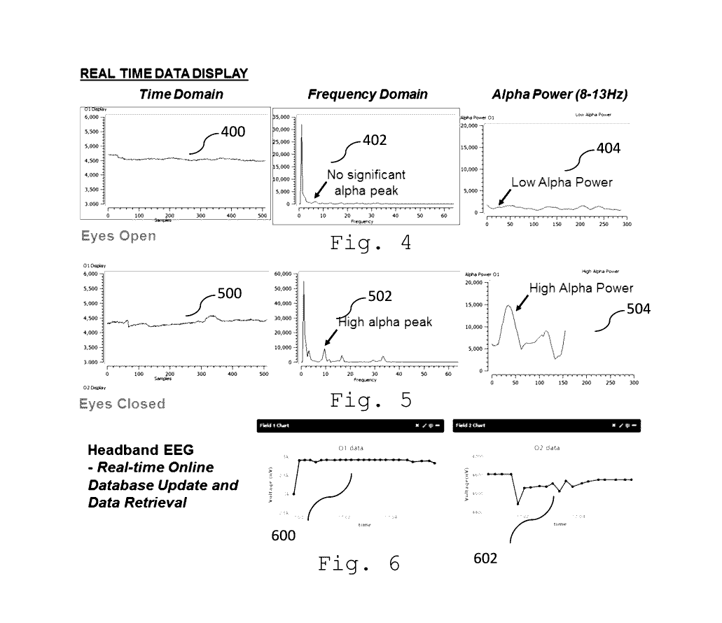

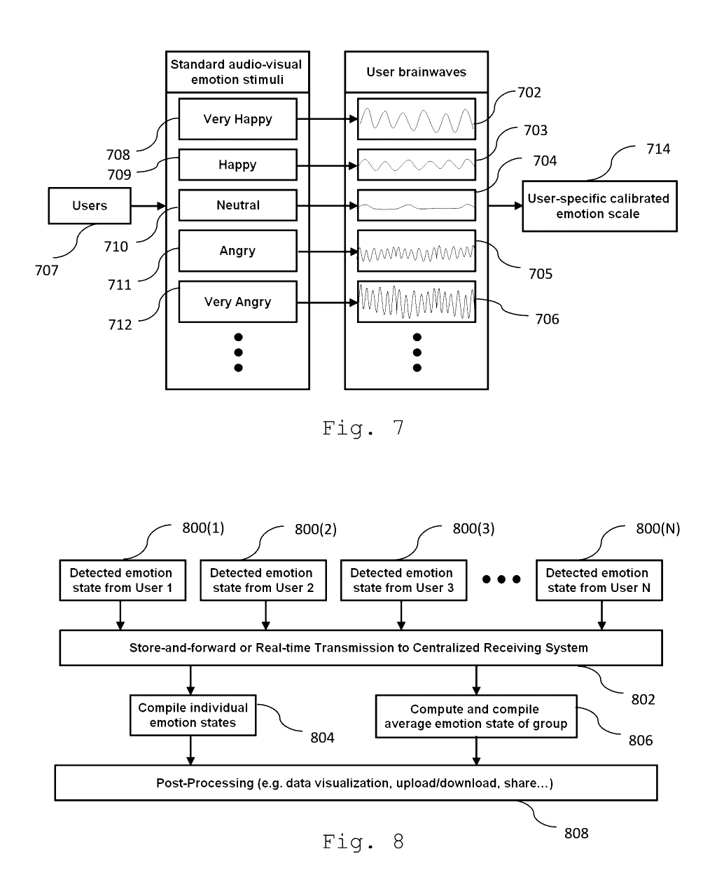

FIGS. 3A)-C) schematic drawings of an example design of a portable EEG device in the form of a wearable EEG device according to an example embodiment, as a headband 300 embedded with monitoring and reference electrodes 302, 304, together with a power board 306 and an amplifier and wireless communications board 308. In this case, the headband design permits shifting of the electrodes 302, 304 from e.g. the frontal positions as illustrated in FIG. 3B), to the occipital positions as illustrated in FIG. 3C), depending on the type of EEG state that needs to be monitored, e.g. frontal and occipital regions correlate with attention and pain respectively. Other adjustability features in example embodiments will be described below, including e.g. with reference to FIGS. 15-21. Data from the EEG device 300 can be wirelessly accessed via a computer/phone/tablet device through a real-time data display (not shown) which plots, for example, the time and frequency-domain EEG information, the power of the targeted brainwave state, as well as processed data that shows the intensity level of the mental state of interest. FIG. 4 shows example plots of the time domain, frequency domain and alpha power (curves 400, 402 and 404 respectively) for an eyes open state, and FIG. 5 shows example plots of the time domain, frequency domain and alpha power for an eyes closed state (curves 500, 502, and 504 respectively). This also applies to other mental states such as but not limited to pain, sleep and anxiety. The data can also be uploaded in real-time onto an online server/database, whereby the data can be remotely accessed anywhere and anytime, as illustrated by plots 600 and 602 in FIG. 6.

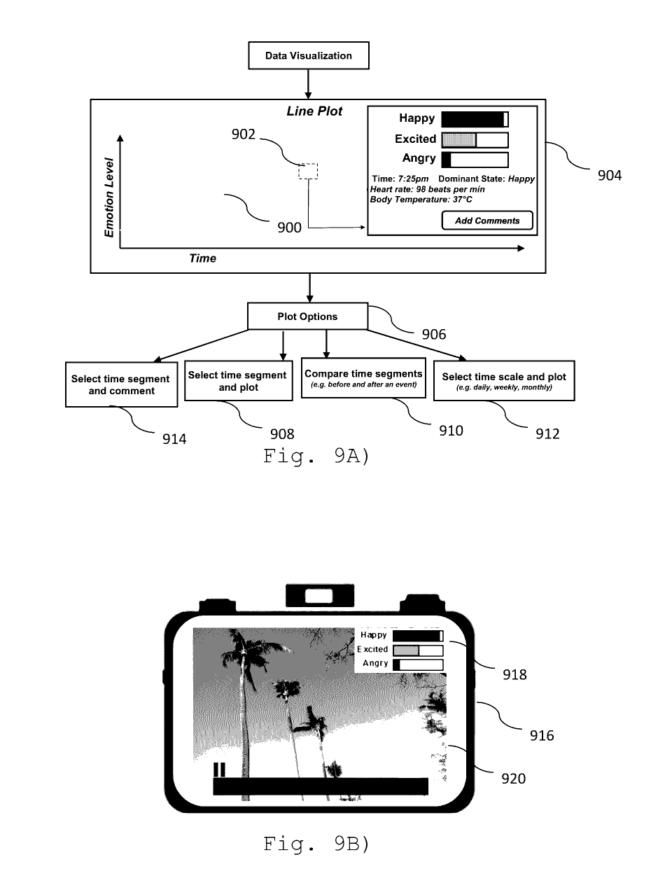

With reference to FIG. 7, brainwaves of interest that can be captured in example embodiments include, but are not limited to, brainwave states of happiness, excitement, attention, motivation, anger, sadness/depression, pain, sleep, anxiety and fear. Different brainwave states tend to show activation in different brain regions and exhibit different waveforms of varying frequency, see plots 702-706. These waveforms include alpha (8-13 Hz), delta (0.5-4 Hz), beta (14-30 Hz) and theta (4-8 Hz). In example embodiments, a calibration procedure/system of the basal level of the user's brainwave states can be performed. The user 707 will for example first be exposed to different audio-visual brainwave stimuli for a particular brainwave state 708-712, and the waveforms that arise from the triggered brainwaves will be detected, see plots 702-706. This procedure allows generating user-specific calibrated brainwave scales 714 to facilitate subsequent brainwave state identification.

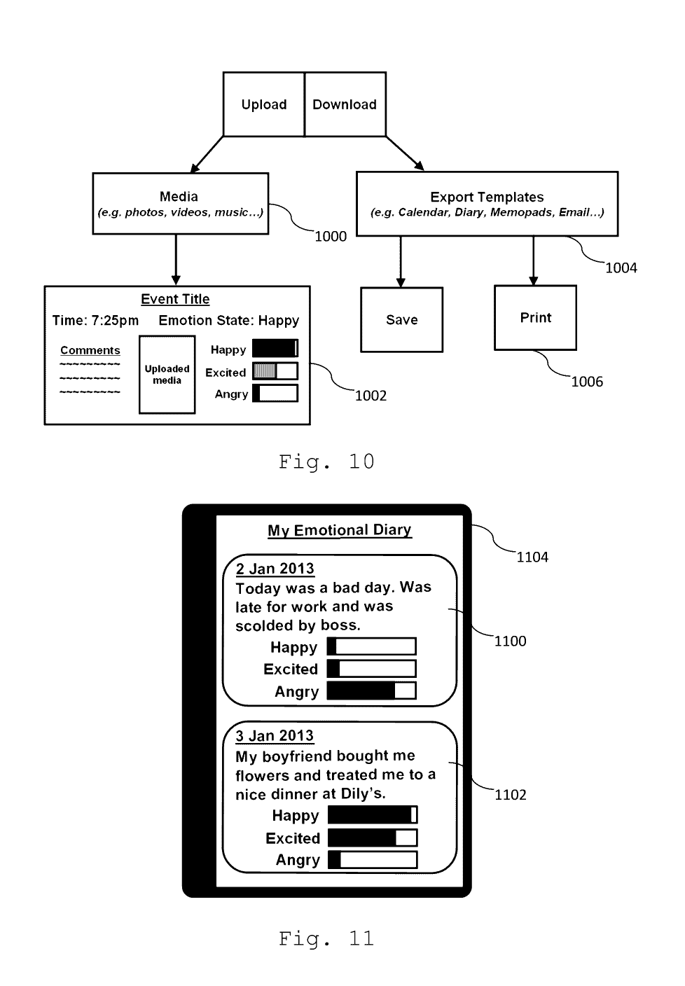

Based on the calibration, brainwave state identification can be obtained and the real-time brainwave state of users 800(1)-(N) can be detected and transmitted to a centralized receiving system 802 in example embodiments, as illustrated in FIG. 8. The transmission approach can either by real-time or store-and-forward (i.e. real-time brainwave data is stored in the electronic device installed with the brainwave identification software and forwarded at a later time to the centralized receiving system when a network connection is available). Brainwave information received at the centralized receiving system 802 can then be stored and compiled as a database of individual brainwave states e.g. 804 or average brainwave states of a group of people e.g. 806 after averaging out according to the number of people participating in the measurement. This brainwave information e.g. 804, 806 can be subjected to post-processing steps 808, which can include, but are not limited to, data visualization, upload/download and share.

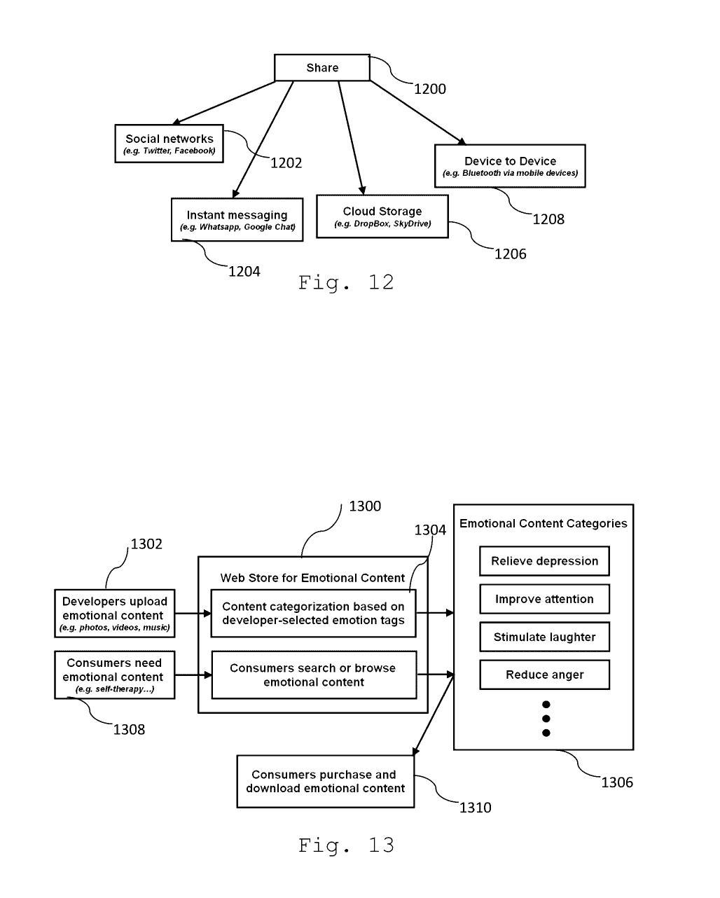

For example, in the data visualization step according one embodiment illustrated in FIG. 9A), the brainwave information can be plotted against time (plot 900), whereby selecting a time segment e.g. 902 of the plot 900 can reveal the brainwave state 904 at the particular time event. The brainwave information can for example be projected on a colored scale to indicate the brainwave levels. The visualization step can allow various plot options 906 for analysis, such as, but not limited to, plotting different time segments of interest 908, comparing different time segments before and after an event 910, plotting continuous real-time brainwave information on various time scales 912 (such as daily, weekly and month). Physiological measurements such as heart rate and body temperature measured by incorporated sensors can also be displayed and correlated with the brainwave data for more meaningful data interpretation. An additional option would be the ability to add comments to selected time segments to provide additional event information 914.

In example embodiments, the EEG-device can be integrated for use with external systems such as cameras e.g. 916 with video function, as illustrated in FIG. 9B). The brainwave information at the particular time event can be recorded during the video recording, and projected 918 on a colored/numerical scale to indicate the user's brainwave levels during a certain experience or activity performed by the user. Such embodiments can e.g. provide video recording of a user experience on the camera screen 920, with display of brainwave information with respect to time.

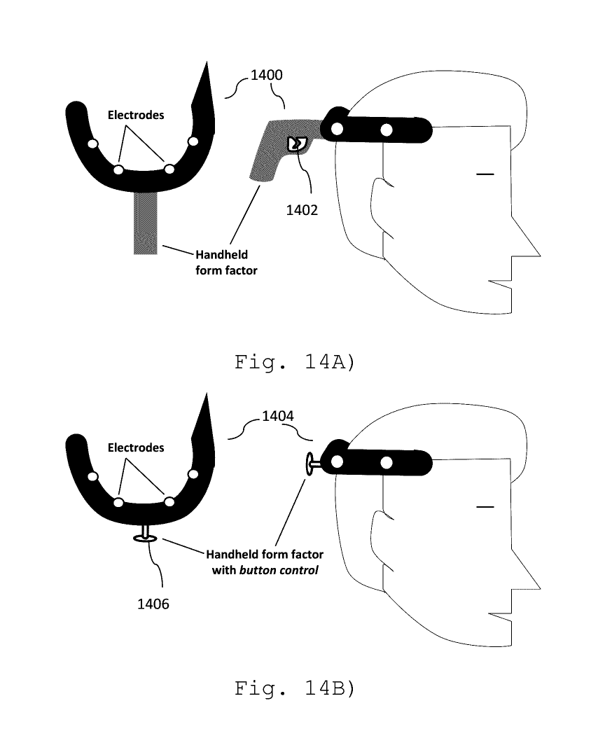

For the upload/download step according to one embodiment illustrated in FIG. 10, media 1000 such as photos, videos and music can be uploaded to the centralized receiving system to provide additional information on the selected time event 1002. In addition, the user can select their desired time events and download them to their personal electronic storage devices, with the option to convert the brainwave data with or without the personalized details into a certain export format 1004 based on different available templates such as, but not limited to, a fixed and/or customizable choice of diary, memopads, calendar, notebook, coasters, gifts etc. For example, a brainwave diary can be printed out 1006 into a hardcopy version of a physical diary, with brainwave information 1100, 1102 coupled to each event in the diary 1104 (FIG. 11). Depending on the user's preference, the downloaded brainwave information may also be automatically exported in e.g. an email format for easy regular sharing and updating. This can provide a digital online capability for the input of personalized documents (comments/photos/files) based on each brainwave information triggered and recorded, and can further allow for the documentation, recording, tracking of events and storage of personal brainwave data collected over a span of a specified period of time in multimedia and printable format. Such an option can be especially advantageous for both personal reference and for institutional research references (e.g. patient records and population-based healthcare monitoring of specific diseased patients).

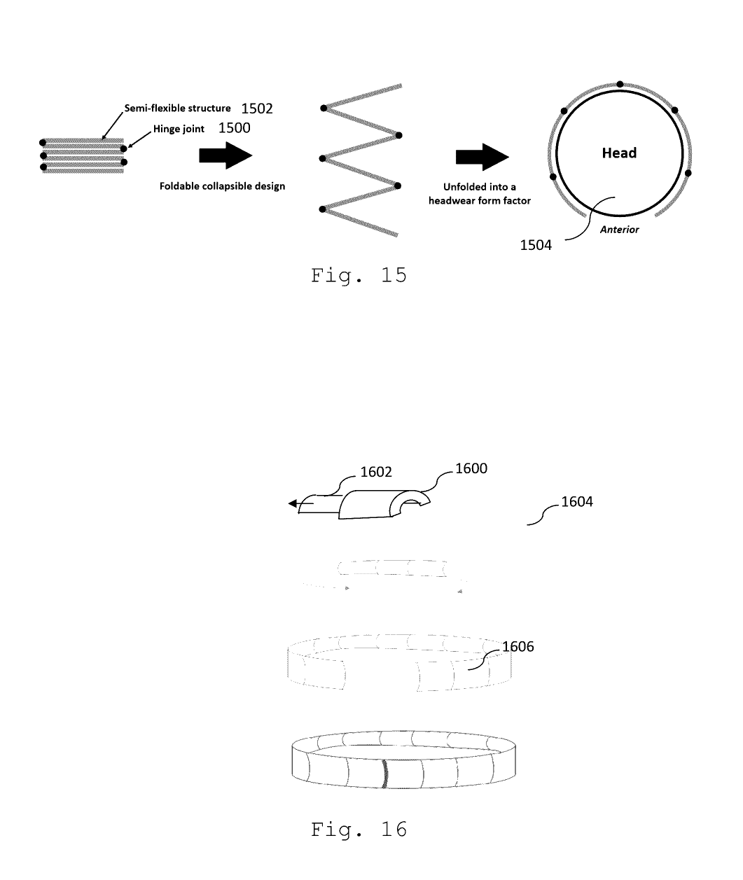

Example embodiments also provide a sharing feature 1200, illustrated in FIG. 12, which allows the user to conveniently share their brainwave information on various platforms such as social networks 1202 (e.g. Twitter, Facebook and Google+) and instant messaging 1204 (e.g. Whatsapp, Google Chat, Skype and email). In addition, the detailed brainwave information can also be stored and shared with selected team members via cloud storage platforms 1206 (e.g. Dropbox, SkyDrive and Box). Apart from sharing the brainwave information using internet platforms, another approach would be to share the brainwave information directly from device to device 1208, for instance, using Bluetooth to transmit brainwave information between mobile devices.

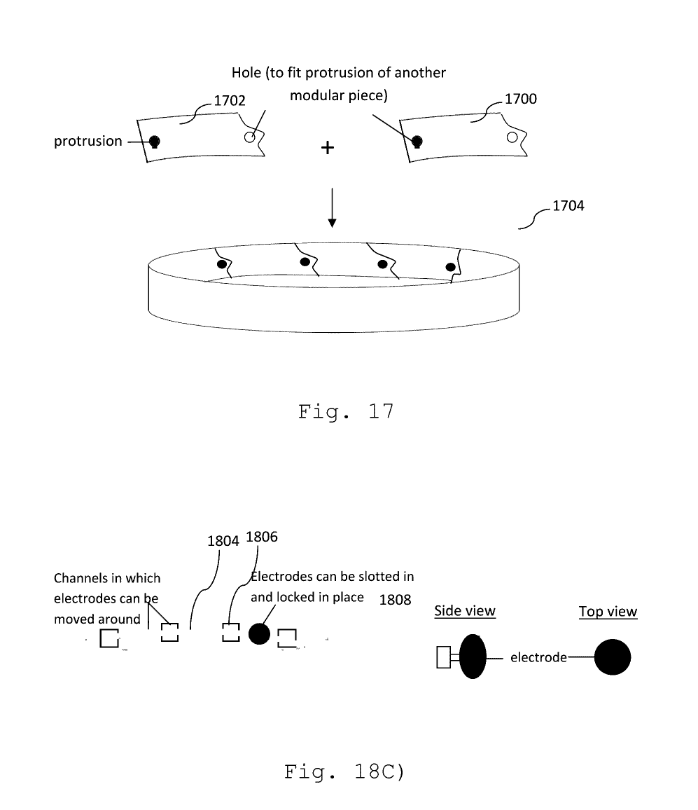

An online web store can be incorporated as part of the brainwave identification software or as a standalone web application in example embodiments, as illustrated in FIG. 13. The online web store 1300 provides a platform for exchange of useful brainwave content, and preferably implements a categorization approach. Developers can upload 1302 and sell their brainwave content (e.g. photos, videos, music and e-books) on the web store 1300. Prior to publishing of their brainwave content on the web store 1300, the developers are asked to categorize 1304, 1306 their content by selecting relevant brainwave tags such as ‘relieve depression’, ‘improve attention’, ‘stimulate laughter’ and ‘reduce anger’. Categorization 1304, 1306 of the brainwave content allows easy search and browsing for consumers 1308 who wish to access brainwave content for various purposes (e.g. self-therapy and anger management). Upon finding the relevant brainwave content, the consumers will then be able to purchase and/or download the desired content, as indicated at numeral 1310.

The portable EEG device in example embodiments is preferably designed in such a way so as to ensure maximum comfort without compromising the signal quality of the brainwaves received from the brain. The form factor of the device can be modified and/or customized to suit individual use-cases and/or personal preferences. In the following, example design option for different embodiments will be described.

Single and Non-Modular Piece With a Fixed Structure

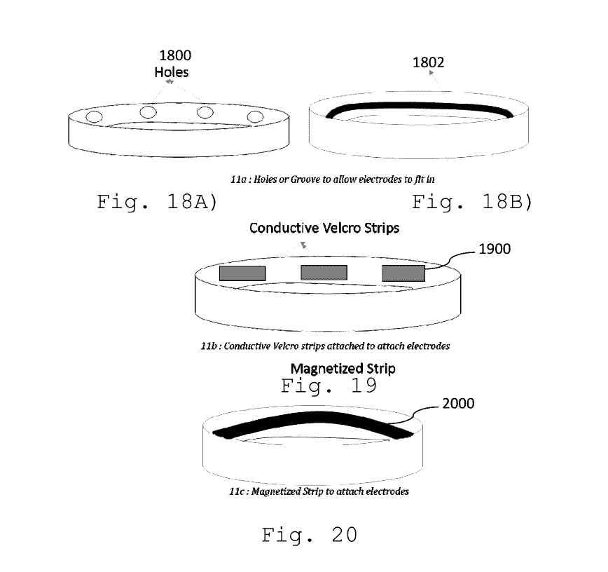

The portable EEG device can exist as a single and non-modular piece with a fixed structure—either in the form of wearables such as, but not limited to, headwear, forehead patch, accessories etc., or can exist in the form of a hand-held device for transient usage. The latter can come in a variety of shapes such as but not limited to a pistol-like design etc. FIGS. 14A) and B) show some examples illustrating form factors of the brainwave-sensing device existing as a handheld system. In the pistol-like design 1400, to start and stop brainwave measurements, the user will pull the trigger 1402, while on a piston-type design 1404, the user pushes a push button 1406.

Collapsible Feature

Collapsibility of the portable EEG device allows the device to be folded into a smaller size without compromising structural integrity. Collapsibility may for example be provided, but is not limited to, having hinge joints e.g. 1500 between semi-flexible structures e.g. 1502 which, from a compact folded state, can be unfolded and affixed at the joints to fit comfortably against the head 1504 when in use, as illustrated in FIG. 15. It will be appreciated that the “collapsible” feature can be provided by other flexible bendable structure(s) and/or foldable structure(s).

Telescopic Design

A telescopic design of the portable EEG device can e.g. have sliding sections e.g. 1600, 1602 which can fit into each other. The sections 1600, 1602 of the device 1604 can slide so as to allow comfortable fitting on heads of all sizes as well as to allow compact storage of the device when not in use. The design, as shown in FIG. 16A)-D), further allows additional sections e.g. 1606 to be used to allow for extension of the device 1604 and/or to accommodate other electrodes or sensors so as to provide additional features. For easy storage, the device can slide into its most compact form. The cross-section of the portable EEG device in the telescopic design can be, but are not limited to, circular or rectangular cross-sectional shapes.

Modular Design

The portable EEG device can comprise of multiple modules which can be disassembled, then refitted or combined with another module. This can come in the form of, but is not limited to, press-fit, lock-fit, hook-like type of individual modules e.g. 1700, 1702 that can be adjoined together to form the device 1704 and to create unique shapes and designs. Such a design preferably places no limitation on the variation of head sizes and allows disassembly for compact and easy storage.

Extendible/Attachment Design

The portable EEG device can have designs to allow for flexible placement of electrodes or other sensors. This preferably allows the addition of electrodes to the device to provide users with a broader range of brainwaves information at different parts of the brain; or the addition of sensors such as heart rate or temperature sensors for measurement of other physiological signals. This expands the scope of use of the device in its ability to correlating brainwave data with other vital signs.

The various device designs described above can exist individually or be combined with any of the various designs for different use-cases.

The portable EEG device in example embodiments comprises of dry conductive electrode(s). The device preferably has multiple electrodes which can be configured in multiple ways to allow for different use cases. The electrodes can be fixed as per an original or default position on the device, or can be adjusted and moved along the form factor of device, or removed from the original or default position and repositioned onto desired area(s) on the device. Additional electrodes can be placed onto desired areas on the device.

The electrodes used in example embodiments of the device are dry and ensures greater convenience which can be used directly without requiring application of gels onto the electrodes in order to provide a conductive medium for brainwave sensing. Such dry electrodes can preferably collect data independent of hair length and thickness and compensate for varying scalp conductance.

Flexible Placement of Electrodes

The electrode(s) can be placed anywhere along the portable EEG device in example embodiments to advantageously allow for collection of data from various parts of the scalp to allow for varied analysis. By allowing flexible placement of the electrode(s) for measurement of brain activity anywhere along the scalp, example embodiments can provide users with a variety of different brainwave information which can range from sleep tracking to anxiety monitoring to measurement of concentration levels.

Moreover, while most existing EEG devices have reference electrodes which are fixed, the portable EEG device in example embodiments allows the flexible placement of the reference electrodes. Preferably, the positions of the reference electrodes are symmetrical in nature.

There are several ways in which the electrodes can be attached to the portable EEG device in example embodiments. In one example, the electrodes are attached using holes e.g. 1800 or grooves 1802 located at suitable locations along the device to allow the electrodes to be fitted therein, as illustrated in FIGs. 18A) and B). Channels e.g. 1804 with slots e.g. 1806 can be implemented in one embodiment, to slot in and lock in place electrodes e.g. 1808, see FIG. 18C). Another way of attaching the electrodes on to the device would be to use conductive Velcro strips e.g. 1900 which would help transmit the information picked up by the electrodes into the main circuitry (not shown) of the device, as shown in FIG. 19. Another way of attaching the electrodes on to the device would be to magnetize the electrodes and the device with opposite polarities, for example incorporating a magnetized strip 2000, and thereby providing the flexible electrode placement, as shown in FIG. 20. The impedances of the electrodes and their attachment points would preferably also be matched to ensure efficient transfer of data with minimal loss.

Similarly, the reference electrodes are preferably not fixed to a particular location on the head in example embodiments, and can be adjusted to match the use case, e.g. if the use case is focused on the left hemisphere, the reference electrodes can be shifted to the left side of the head to capture differences in brainwave signals between different regions of the left brain.

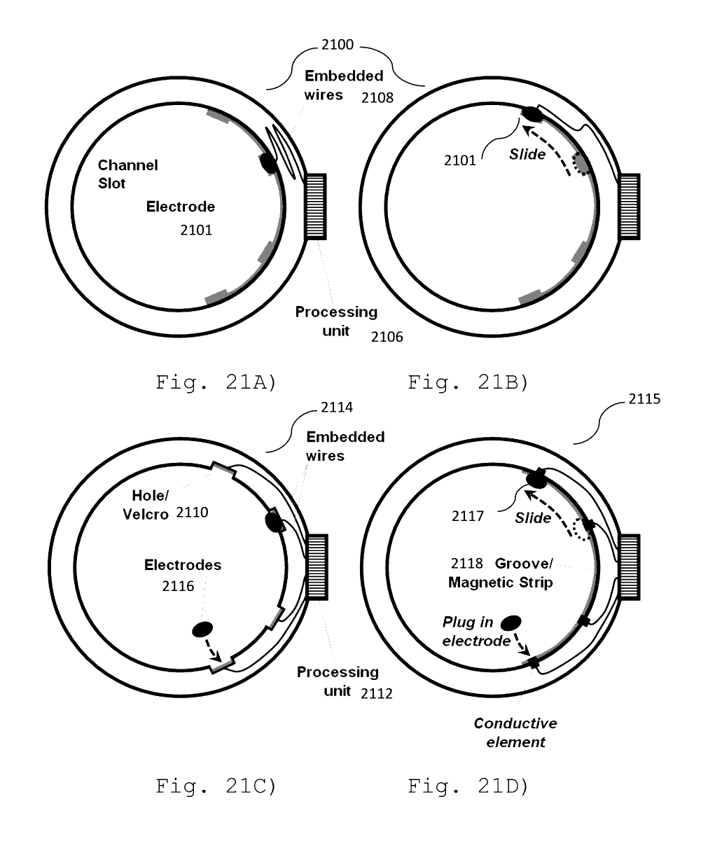

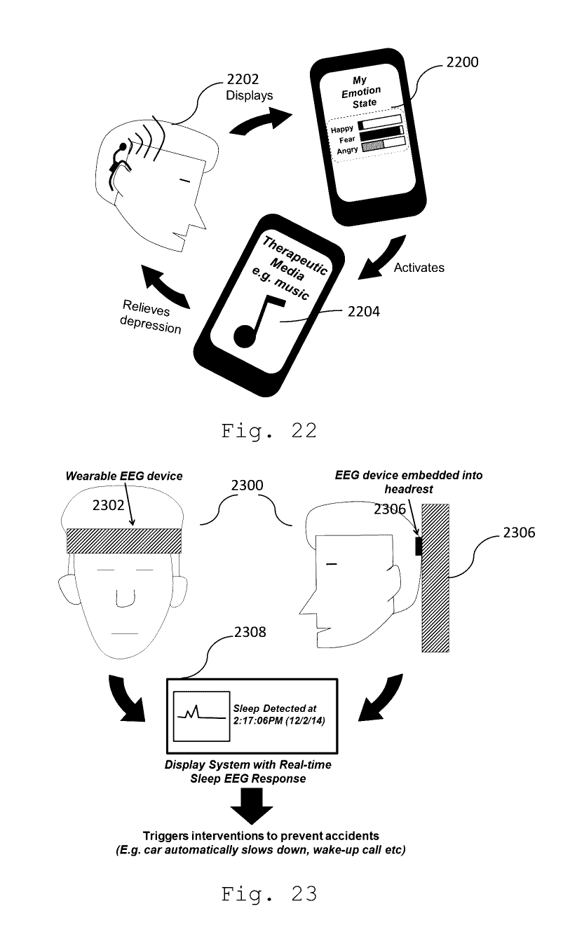

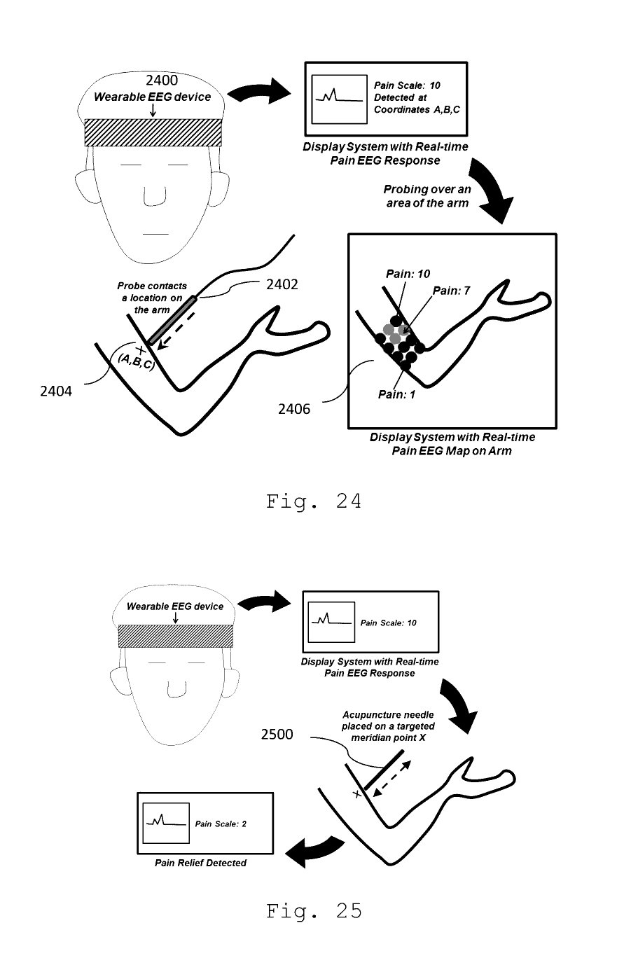

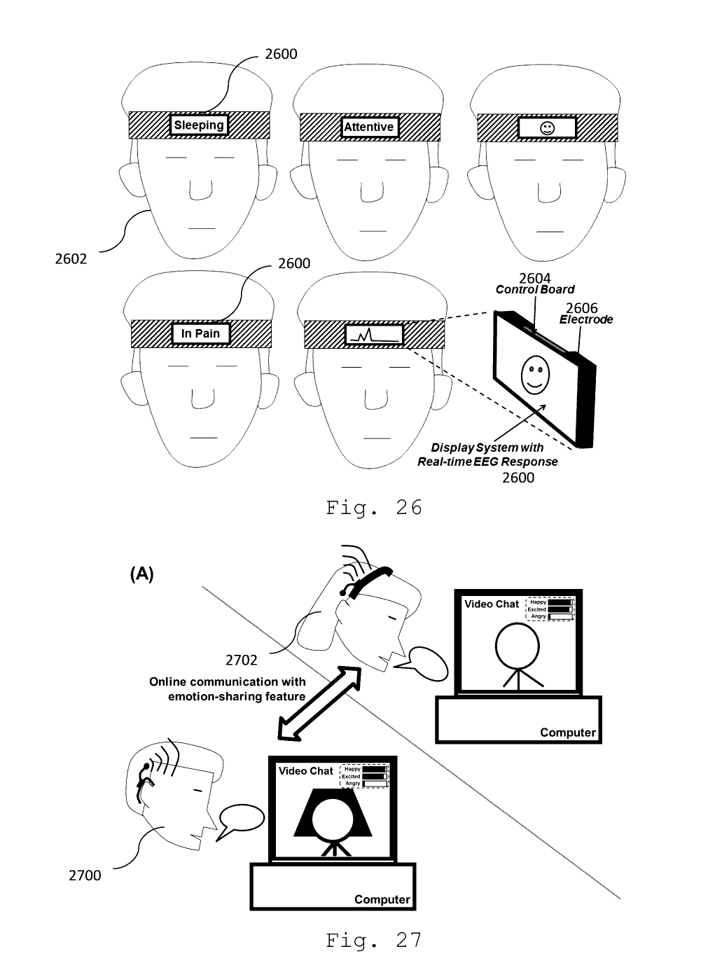

The electrical connections (e.g. wires) between the electrode(s) and the processing unit can be embedded within the form factor casing of the device 2100 in example embodiments, for example such that the electrode e.g. 2101 can slide and lock onto different slot locations e.g. while maintaining connections with the processing unit 2106 via extendable wires e.g. 2108, as illustrated in FIGS. 21A) and B). Alternatively or additionally, as illustrated in FIGS. 21C) and D), the electrical connections (e.g. wires) between conductive elements (e.g. conductive velcro 2110, conductive gold alloy strip etc.) and the processing unit 2112 can be embedded within the form factor casing of the device 2114, 2115. The electrical connections can be fixed within the form factor in such embodiments, in a distributed array, such that the electrodes e.g. 2116, 2117 can e.g. be plugged into a hole, onto the conductive velcro 2110, into a groove or onto a magnetic strip 2118, and thus achieving electrical connections to the processing unit 2112 via contact with the conductive elements. The electrodes e.g. 2117 can also be adjusted along e.g. the groove or magnetic strip 2118 to attain electrical contact with other conductive elements in different locations in example embodiments.

The utility of brainwave data for various applications according to example embodiments has been broadly categorized into (a) communication (such as, but not limited to, social, healthcare, sports and medical purposes) (b) products, services and entertainment (such as, but not limited to, dining, gaming, movies, remote control gadgets/toys, driving etc.) and (c) rehabilitation. Some examples of how the collected brainwave data can be used include, but are not limited to, self-improvement, remote brainwave-detection and brainwave-sharing via digital platforms, brainwave-tagging of various digital media, centralized brainwave data compilation system, brainwave-monitoring for social and healthcare reasons, brainwave-based rating of products and services, brainwave-targeted advertisements, brainwave-induced drug release, communication via brainwave-engagement with illness-stricken parties who are in comatose/vegetative state, stroke and/or unable to communicate or express themselves, as well as brainwave-controlled social robots. Some example applications will now be described in detail as follows:

Brainwave-Detection and Monitoring

The complexity of human minds evolves from the paradigm of brainwaves and feelings and there could be useful data that we can potentially extract to be put to use for both personal and collective benefit. To understand how humans are affected by brainwaves, how the brainwave state can potentially impact on humans' lives and how to use brainwaves to value-add to daily live, example embodiments of the present invention allow for brainwave-detection and monitoring. This can be accompanied by the use of a system/a software such as, but not limited to, the systems/software described above, where the user's brainwave state can be tracked, recorded over a certain period of time, and compiled together as a graphical and/or statistical summary at the end of a specified duration (e.g. at the end of each week, month or year). This brainwave information can then be stored electronically and/or downloaded for other uses as described below:

(i) Brainwave-Detection and Monitoring For Self-Improvement

Example embodiments can be applied to serve as an assistive tool to help individuals be more aware and in control of their own brainwave states, acting as a self-check device to maintain a healthy state of mind. Sometimes, an individual does not pause to think and respond almost immediately to a particular behavior. Individuals may let their emotions overrun which in turn leads to regretful actions. This lack of awareness can often result in poor interpersonal relationships, misunderstandings and other associated problems such as depression. Example embodiments can provide self-therapy for maintenance of a healthy mind and well-being, hence maintaining a happier society on the larger scale.

The application (‘app’) or device in such embodiments displays the user's brainwave state and the user will be notified only when the brainwaves go beyond a certain emotional level that is calibrated as ‘healthy’. At this point, for example in state 2200 dominated by fear, the user 2202 will be given an option to activate a therapeutic audio-visual solution 2204, such as, but not limited to, music, images, readings, videos and games. The list of different therapeutic solutions can be personalized based on different brainwave states. For example, an angry person can be notified of his anger state, prompting him to play a self-healing soothing music for calming effect. A sad person would be prompted by a selection of funny videos to induce laughter. This methodology in example embodiments would be most useful when the person is constantly on the move. In addition, a computer-literate user can utilize the online software to customize advertisement pop-ups based on brainwave that is being expressed at that point of time. For example, when the computer-user feels stressed, he can pre-select from his list of favorite websites that can provide him instant relaxation. This application can be a potential tool that psychiatrists can recommend to their patients who suffer from some level of brainwave dysfunction such as depression and anxiety.

Example embodiments can also be applied towards anxiety sensing, particularly for, but not limited to, athletes, in which anxiety has been reported to influence athletic performance (Hann, Y. L. (2000). Brainwaves in sports. Champaign, Ill.: Human Kinetics). For instance, a wearable EEG device objectively detects the level of anxiety of the athlete in real-time, and the coach is then notified of the athlete's anxiety level. If the anxiety level is high, the coach can then make a decision to implement interventional measures (e.g. stretching exercises for athlete, pep talk etc.) to calm the athlete, prior to the commencement of the competition. The coach can subsequently check the athlete's anxiety level again to confirm that the athlete's anxiety level has dropped to an acceptable level for optimal performance in the competition. Apart from anxiety, example embodiments can be implemented for attention quantification during execution of certain maneuvers during static (e.g. air rifle) and dynamic (e.g. soccer and tennis etc.) sports, which would advantageously generate quantitative information for athletic performance management and optimization.

Sleep detection is another possible application of embodiments of the present invention, whereby the user 2300 can don a wearable EEG device 2302 during driving or an EEG device 2304 can be embedded into the user's headrest 2306, see FIG. 23. Detection of sleep 2308 during driving by the EEG device 2302, 2304 can help to trigger interventions, such as automatic slowing-down of the car with hazard lights on and wake-up calls, to prevent accidents on the road. Example embodiments can also be used for sleep quality monitoring, whereby a wearable EEG device can record the brainwave states during the user's sleep, in order to determine sleep quality. Thereafter, the user receives a post-sleep objective assessment of his/her sleep quality. If the sleep quality is poor, the user can test interventional measures (e.g. soothing music, room lighting etc.) and then check with the EEG device again to determine whether the measures are effective in improving sleep quality. This intervention can also be implemented real-time by e.g. triggering room speakers to turn on soothing music in response to detection of poor sleep quality. Also, the system can be modified to measure the sleep quality within a preset sleep time; thereafter, once the sleep time is achieved, the system will trigger a wake-up call so that the user wakes up in time, feeling refreshed.

(ii) Brainwave-Detection and Monitoring For Healthcare

The applications of brainwave-detection and monitoring can be extended in example embodiments to healthcare and medical treatment, which is particularly useful for monitoring the mental states of people with brainwave irregularities, resulting in behavioral issues and challenges with interpersonal relationships. This brainwave information is most often important to, but is not limited to, parents, guardians, counselors, healthcare personnel or medical doctors to track the real-time brainwaves of their child, client and/or a group of people concurrently. For example, psychologists, counselors and psychiatrists could monitor their clients' disorder/condition (such as autism, bipolar disorder, ADHD etc.) in real time from their workplace, enabling tracking of the progressive condition of their client and to administer appropriate treatments to improve the patient's brainwave states.

This advantageously provides greater convenience and improved quality of treatment for their clients upon meeting. Parents or guardians could monitor their child more closely, who could be suffering from excessive stress levels or psychological trauma due to child abuse or bully by others; as well as elderly in the family who are struggling with managing ageing illnesses such as Alzheimer disease as well as the associated emotion-related issues. By remotely monitoring the brainwave states of these people in real-time, their mental health can be diagnosed beforehand, allowing others to empathize with their condition and provide adequate care for them. This application also has potential benefits especially in terms of emotional rehabilitation, for instance, helping prisoners rehabilitate through anger or violence management.

This application can be extended in example embodiments for use on a larger scale, for detecting and monitoring the brainwave states of patients in a center, hospital or institute. One such example is the mental institute, where a large group of mental patients with emotional dysfunctions need to be closely monitored. For this application, preferably all the brainwave data collected from the individuals (and their respective locations) can be channeled to a centralized receiving system of an example embodiment, such as described above with reference to FIG. 8, where the healthcare personnel can now monitor and track the brainwave state of all patients with greater convenience and provide more prompt and quality care for these patients. This could be further coupled with the closed circuit television (CCTV) to double up as a monitoring device, above that of the display of the threshold of brainwave levels of each individual.

In addition, the application can be implemented in different embodiments for pain localization, as illustrated in FIG. 24. For instance, a wearable EEG device 2400 such as a headband or watch etc. worn by the patient can be used in conjunction with a probe 2402, whereby the probe 2402 (held by the clinician) contacts a certain point on the body for example, arm 2404. The magnitude of pain recorded by the device 2400 is then registered to the 3D coordinates of the probed point. The clinician subsequently contacts other points on the arm with the probe, such that a pain-EEG map 2406 can be derived, which can help to objectively localize the pain and non-pain regions. Also, the system can be modified for use as a pain monitoring system for objectively assessing the efficacy of pain-relieving methods (e.g. acupuncture 2500, medications etc.) in patients, as illustrated in FIG. 25. In a similar fashion, the brainwave information obtained from the device can also be used to control the rate of drug delivered to patients who are suffering from pain for example, after a surgery.

(iii) Alternative Form of Communication Via Brainwave-Detection and Monitoring

This application in example embodiments explores the potential of allowing people, who has limited ability to express themselves either through facial expressions, verbal and/or physical gestures (either due to, but not limited to, stroke, cerebral palsy and multiple sclerosis) to communicate with others through their brainwave signals that they express. This is particularly useful as an alternative form of communication for, but not limited to, the patient's family members as well as to the patients themselves in helping them to relieve their frustrations in not being able to communicate whilst people now can better empathize with their feelings and intentions.

Advantageously, this application can potentially help patients who are in a vegetative state regain consciousness earlier by identifying the most optimal form of stimulation through detection of a change in their brainwave state. For example, a common technique that family members would do for the vegetative patient is to provide stimulatory experiences of their senses that mimic an old memory. Family members of patients would now be able to know if the therapy is helpful based on a change in response of the patient. Rather than doing repetitive stimulation e.g. playing of a certain musical piece that they may not respond well with, experimentation with other forms of stimulation could possibly be more useful. This way, the brainwave state of a comatose patient may be tracked, displayed and reviewed over time by the family members to provide a form of two-way communication via brainwaves. The brainwave information can be potentially combined with electrocardiography (ECG) data to provide a better gauge of the response of the comatose patients, which may be triggered by family members' stimulatory methods. In addition, the EEG data can be directly fed to, for example, a forehead display 2600 on the patient 2602 (or another device such as a watch or handphone etc.), such that any EEG state (e.g. attentive, sleeping, in pain, happy) that is detected by the device can be immediately shown on the display, for example in the form of, but not limited to, words, graphical plots, numerals and emoticons, as illustrated in FIG. 26. The display 2600 can incorporate a control board 2604 and electrode(s) 2606. This direct EEG-to-Display system in an example embodiment potentially allows for intuitive face-to-face communication between vegetative patients and family members.

Apart from communication, such embodiments could potentially aid in detective or police investigations especially when victims are traumatized and unable to communicate normally or the person under interrogation does not cooperate. In addition, this application is also not limited to only brainwave-detection and monitoring but can also be extended to assist in the rehabilitation of patients who have temporary loss of functionality in certain brain functions (e.g. speech, planning, memory and motor skills) due to diseases such as stroke, Alzheimer's disease and autism, whereby this application can be used as a form of assessment to evaluate the recovery of the patients. Such embodiments are not limited to the healthcare monitoring of brainwave states but can also be extended for use as devices for disease diagnosis (e.g. stroke, Alzheimer's disease, hypoglycemia, apnea) to facilitate prevention and treatment.

(iv) Brainwave-Detection and Monitoring For Societal or Global Issues

This application of brainwave-detection and monitoring in example embodiments can be implemented on a larger scale to obtain statistical data of a larger group of people, for the understanding of population-based behavior or issues and/or to help people make more informed choices. For instance, understanding the general brainwave state of a group of people in a discussion within one room, may help a person to decide whether to join the particular discussion group. As another example, Understanding the general brainwave state of the population residing in a country can more accurately measure the happiness index of a country, thereby aiding in the decision-making process of whether to migrate to the particular country.

Using brainwave-detection and monitoring could be performed with high accuracy in example embodiments by directly measuring the brainwaves of a large population of people with minimal disruption to the daily lives, where this information can then be collected and compiled through a centralized system. Other studies that require input on a nation/country and/or global level could also use embodiments of the invention for accurately tracking of the brainwaves of people, thereby unraveling important issues such as Gross National Happiness. Other possibilities of this application in example embodiments can range from location-based tracking of the brainwaves of different groups of people (indoor and outdoor monitoring), tracking happiness levels of citizens on a country and global level (rather than happiness index), to tracking attention levels of people in a class or conference session.

(v) Brainwave-Detection and Monitoring For Animals

The real-time brainwave monitoring of brainwaves can potentially be translated for use on animals. Notably, performing animals such as, but not limited to, horses trained for racing or shows need to be kept up to form for optimal performance. The utility of embodiments of the invention can come in handy as it serves as a predictor of performance level prior to the event, hence allowing the owner to strategize and/or prime the animal accordingly.

Brainwave-Sharing

The world is now easily connected by flights, where commute across the globe has become so easy and common. In addition, the world is becoming increasingly digitized with the rise of a digital media and technology that has brought about greater convenience in our daily lives allowing us to communicate with people remotely through a virtual platform. This application in example embodiments serves to further bridge this mode of distant communication, and to humanize digital interactions through our feelings and brainwaves, hence facilitating digital communication and making these interactions feel more like reality, particularly for people who desire more personal interaction with specific individuals on a regular basis but are limited geographically.

(i) Brainwave-Sharing Via Video or Voice Calls

Embodiments of the invention can be designed to improve relationships between specific individuals or groups of people by allowing their true emotions to be reflected during their virtual communications. This can be particular useful for, but is not limited to, those in long distance relationships relying on video or voice calls as their main mode of communication, as illustrated in FIG. 27. By understanding how the other party 2700, 2702 feels, it would help to facilitate communication and minimize the occurrence of unintended misunderstandings, thereby fostering stronger ties between parent-child, couples, friends, business partners etc.

(ii) Brainwave-Sharing Via Short Text Messages

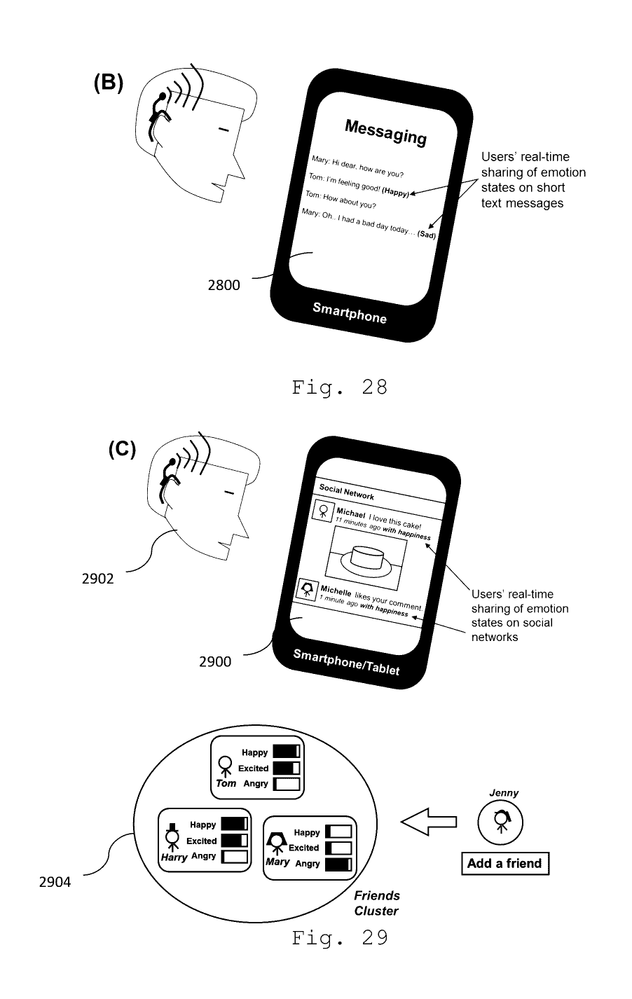

In addition, embodiments of the invention can be extended to group chats where a customized cluster of people, such as family or close friends are given the option to share and track one another's brainwaves via a platform 2800 that could allow simultaneous exchanges of short text messages through web or mobile communication systems, as illustrated in FIG. 28. In being able to keep remotely track of the emotional status of others in real-time, it allows people to understand and care for their loved ones as and when is needed, especially during crucial times such as when the person met with unforeseen mishaps or is feeling emotionally down.

(ii) Brainwave-Sharing Via Social Networking Platforms

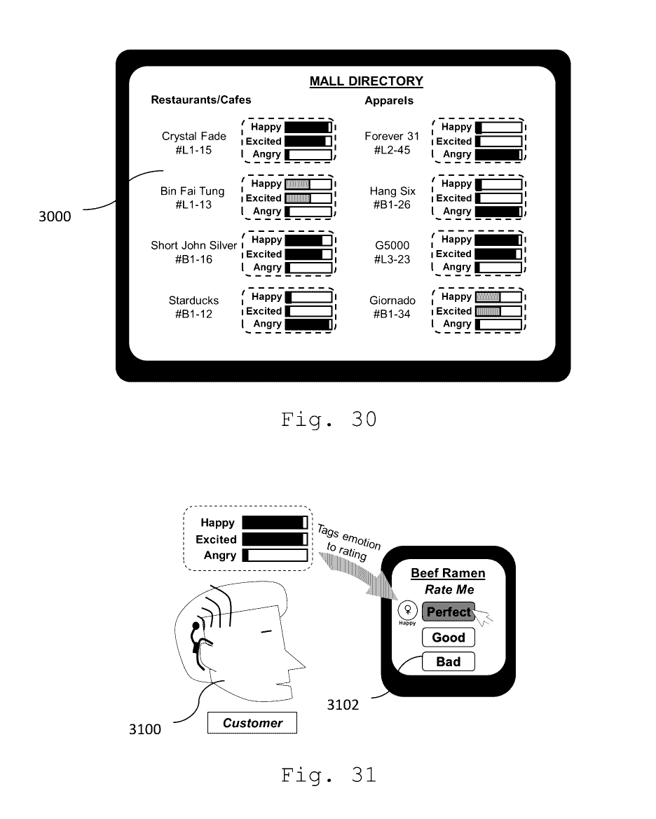

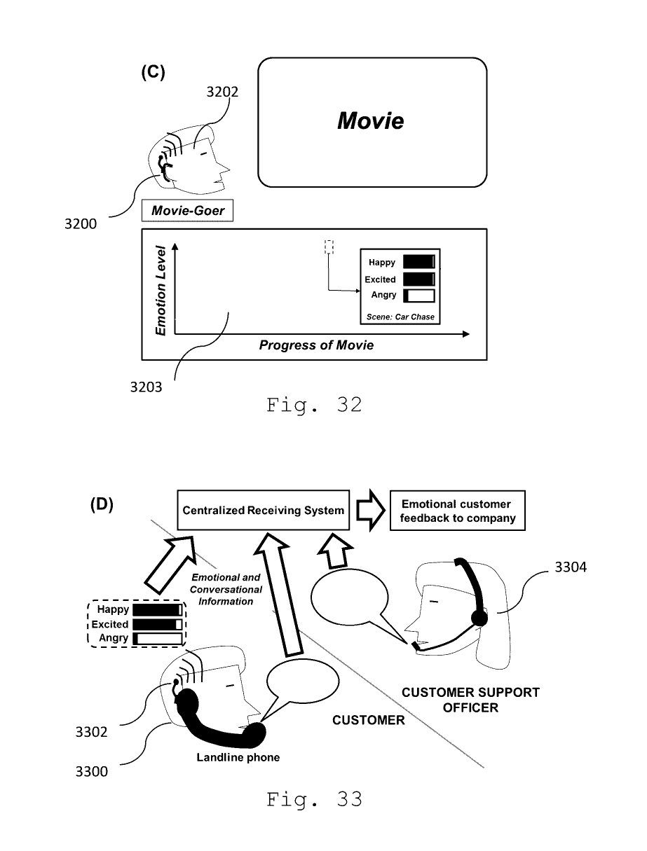

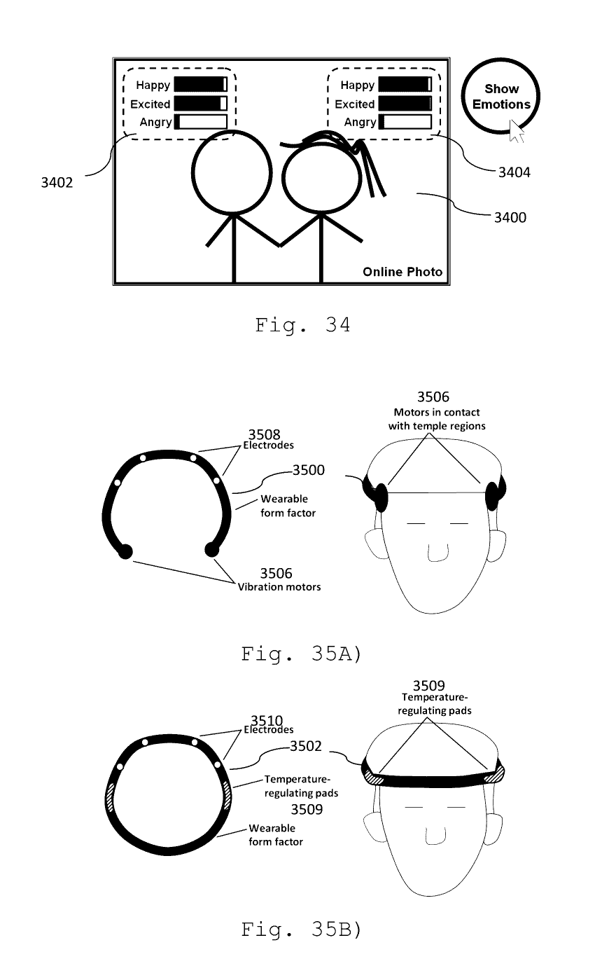

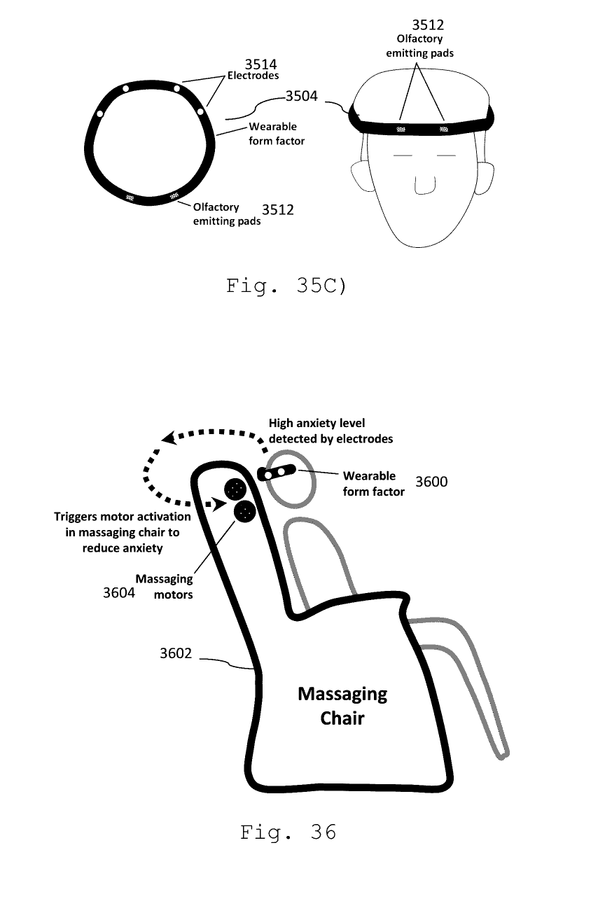

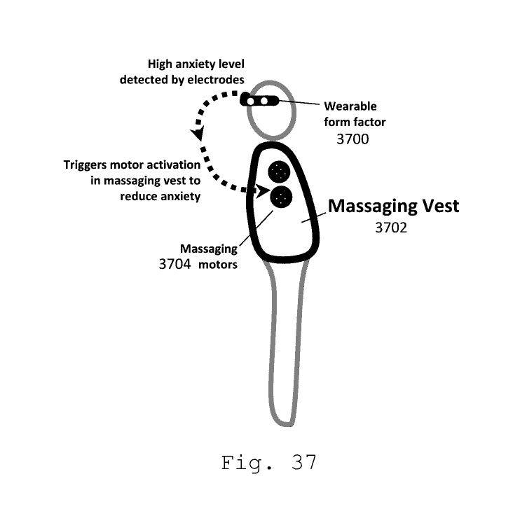

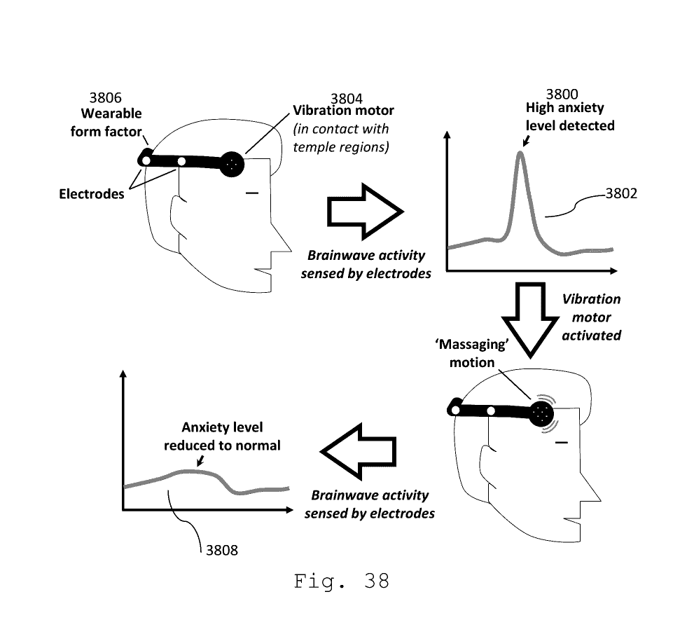

The application of brainwave-sharing in example embodiments could be further applied to social networking platforms 2900 and through gaming interactions, where users are given the privacy option to upload and share their brainwaves, as illustrated in FIG. 29. For example, but not limited to, a Tweet, Facebook ‘Like’, the act of uploading a photograph, the signing in to a location, the reflection of a gaming score can each be coupled with the experience of the user 2902 through the display of his real-time emotions at that specific instant. This can also be applied for forming and joining friends clusters 2904. The intent is to humanizing this virtual experience and making it a more fun-filled, interactive and realistic.

(3) Brainwave-Based Rating

The brainwave information obtained from each individual can serve as an accurate form of rating for user satisfaction of a product and/or service in example embodiments. Such embodiments can be used in almost every industry where ratings are important for the company to improve their services/products based on the feedback and also to help general consumers to gauge and decide their preferred choice. Real-time or past brainwave-based ratings are applicable for evaluating the standards of an event such as educational talks, classes, exhibits. This rating approach can also be applicable for the rating of a teacher in order to identify how her training should be improved or for identifying top candidates or speakers for certain awards based on the audience's emotions; it can also be used to rate against someone (e.g. a celebrity's facebook page) to better assess his/her true popularity. This approach is not limited to brainwave-related ratings, but can also be leveraged on non-emotional information such as sleep, attention, learning and memory.

This approach of rating in example embodiments can be more useful for experiential-based experiences that involve brainwaves rather than based on an objective rationale, more commonly so when a service is involved. This experiential rating can be used to determine the true satisfaction level of customers as compared to the current system of objectively rating products/services on a numerical scale. Such as tool can help facilitate decision-making of the customers so as to provide them with maximal value and experience, as well as act as a feedback system for the company of interest to constantly seek to improve on their services/products. A centralized system where the brainwave information will be collated, calculated and displayed could be used for rating the individual branch outlets or averaged out as a collective representation of the company of interest. Example embodiments of this application can be widely used and implemented as a rating tool due to its accuracy based on customer satisfaction that it could potentially be used as a compulsory test for rating products and services under the International Organization for Standardization (ISO).

(i) Shopping Malls/Restaurants/Menu/Dishes

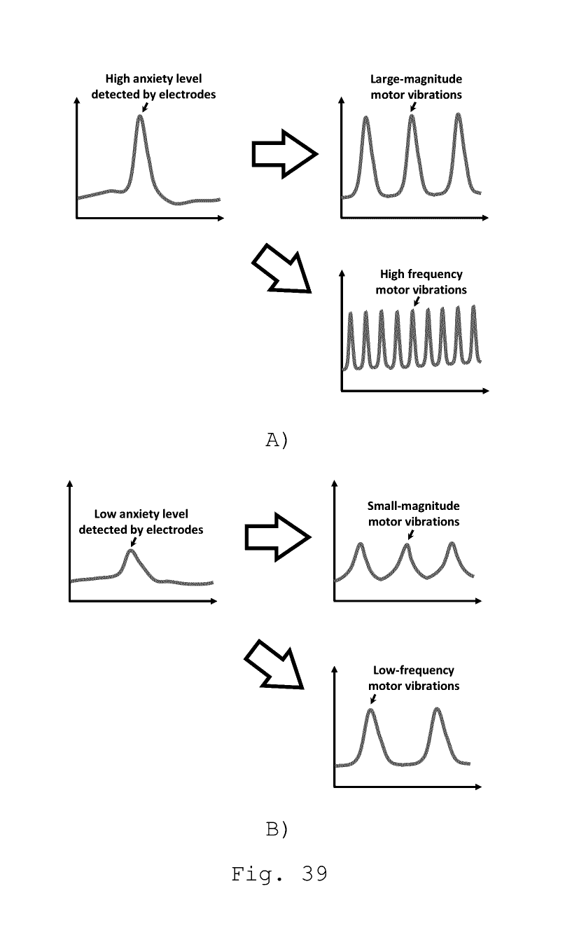

There is a wide range of choices one can make when it comes to a daily affair of dining and shopping. Example embodiments of the invention can expedite the decision-making process for customers during activities such as, but not limited to, dining and shopping. For instance, the directory 3000 of a shopping mall or supermarket would show the real-time average brainwave state of the customers in each store e.g. 3002, as illustrated in FIG. 30. Another example would be that the specific dishes of a restaurant menu would be reviewed at a glance, where real-time or past brainwave rating of each dish will be displayed. In addition, a patron 3100 can also tag emotion of the patron 3100 to rating 3102 of a specific dish food and/or the service of a restaurant after a meal, which would serve as a form of customer satisfaction feedback for the restaurant to act on and improve the quality of their dishes, as illustrated in FIG. 31. Another example is real-time ‘in-meal’ brainwave tracking, whereby the eating process of a customer consuming Ramen noodles can be captured emotionally using the invention, and visually using a video camera. The combination of this brainwave and visual information can provide insights to what ingredients are triggering positive emotions in the customers and hence helps the chef to decide the optimal configuration of ingredients that can bring about the best customer experience. The customer eating of Ramen noodles can be featured on a video recorder to understand at specific times of tasting their dish, whilst the real time brainwaves are being displayed.

(ii) Entertainment

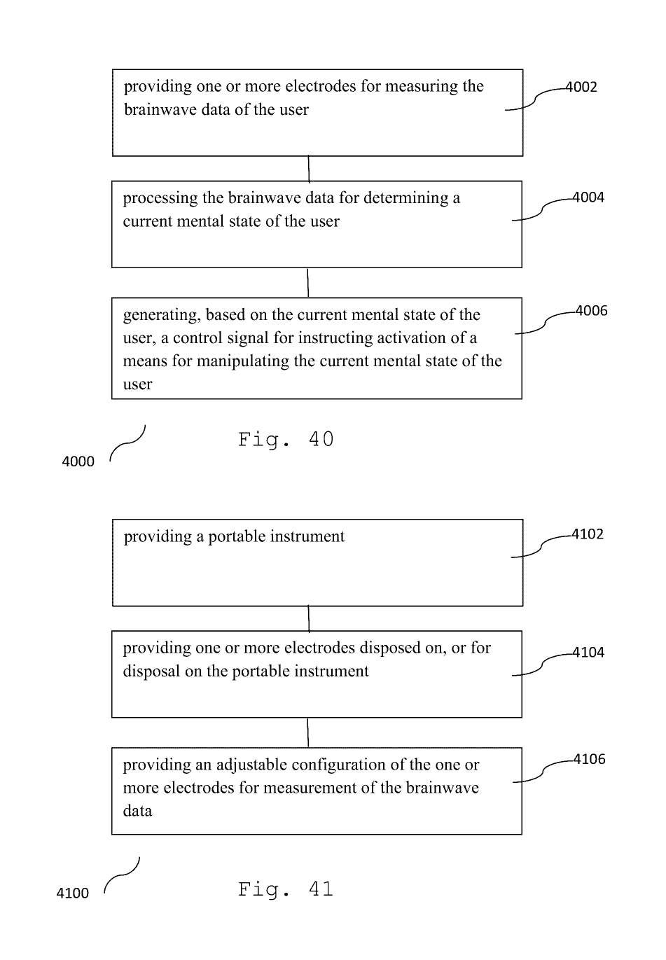

Embodiments of the invention can be incorporated into 3D glasses or used as an independent device for movie-goers. The device 3200 can capture the movie goer's 3202 brainwaves throughout the course of the movie, see plot 3203, which would potentially serve as important feedback information for movie producers on the downsides and upsides of their movies and can also act as a form of rating to aid other movie-goers to decide which movies are worth watching, as illustrated in FIG. 32. Synopsis can be written based on the real-time emotional expression of the audience, thus providing a more accurate and detailed review and for users to look out for specific interesting scenes. This form of emotional customer experience is not limited to movies, and the same concept can be applied to hotel stay, gaming, performance, TV shows, radio station, songs/music or even websites. For instance, the emotional browsing experience of a website can be tracked through the invention and the average visitor brainwave rating can be included as part of the page ranking algorithm in search engines, such that websites with great emotional ratings will appear top on the search page. Online streaming of songs on the websites of radio stations could also display the rating of each song that is being played.

(iii) Customer Service Support Experience

One usually has to visit the store directly or call up the customer support hotline, in order to obtain assistance for certain product or service. Embodiments of the invention can be incorporated into every store or even into every household landline as illustrated in FIG. 33, whereby the customer 3300 will be prompted to connect the electrode 3302 whenever being served over the line. The brainwave profile of the entire conversation between the customer service officer 3404 and the customer 3400 will be recorded. This form of brainwave feedback can help companies maintain a high quality of customer service.

(4) Brainwave-Tagging For Personal and Security/Detective Use

Brainwave-tagging in example embodiments is a method in which the user's brainwaves can be captured and given the option to be reflected. Apart from visual capture of information through photo-taking, the brainwave states of the people being photographed may also be captured at the same time through the invention. Once the photo 3400 is uploaded, the brainwave states 3402, 3404 of the photographee can be displayed alongside or revealed upon clicking of a button or placing the mouse cursor over the photographee's face, as illustrated in FIG. 34. Similarly, example embodiments can be applied to videos or even CCTV, where real-time brainwave tracking of people within the video can be performed to understand their brainwave states at the instant at which they exhibit certain behavior or actions. An example would be the combined use of brainwave-tagging and motion-sensing technologies in CCTV such that it can track user brainwaves and body languages at the same time. Such embodiments can help facilitate police interrogations of potential suspects or even during security checkpoints. For example, at highly sensitive areas where high security is required, compulsory utility of this device within the entire building could be used to monitor the entry of suspicious people by observing their body language and behavioral signals, whilst simultaneously detecting and monitoring their associated brainwaves. Such embodiments could also be used for remote healthcare monitoring and detective purposes. Moreover, such embodiments can be combined with location-based tracking technologies such as GPS, which can be particularly important for connecting with, but not limited to, family members, psychiatric or autistic individuals, as well as for social purposes in enhancing the understanding one's experience in certain locations.

Brainwave-Induced Applications

Embodiments of the invention can leverage on the users' own brainwaves to run a certain useful application.

(i) Brainwave-Induced Advertising

Advertisers can categorize their product/services by having a particular brainwave state tagged to it according to example embodiments, allowing the product/service to pop up according to the user's brainwave state. This could be implemented in conjunction with the online web store as mentioned above. For example, a depressed person may trigger the display of an online advertisement offering psychological hotline assistance or even shopping therapy for an upset individual may be deemed helpful. In addition, a happy person may be prompted by celebratory advertisements that sell items which of great interest to him/her.

(ii) Brainwave-based Games

Games can be designed to incorporate the user's brainwaves in example embodiments, with the option to display, monitoring, share and also act as a feedback for controlling part-of or whole of the running of the game. This would humanize the game characters or activities, making it more interactive and realistic. Current games typically sense the physical movements to power the games, but using brainwaves with games to induce a certain function in example embodiments can help enhance the user's experience. For instance, a gamer who exhibits an excited brainwave state can trigger a special attack move for his character to defeat the opponent.

Brainwave-based games according to example embodiments could also be useful for the rehabilitation of people suffering from brainwave problems (e.g. depression), for instance, a smartphone game may require the user to be happy everyday in order for him/her to earn free daily virtual rewards (e.g. virtual coins) to help them proceed further in the game. This would encourage the user to maintain a happy state everyday and any progressive improvement in his/her mental states can be tracked over time using the aforementioned software. In addition, the brainwave information can also be used as a form of competing element in a game, whereby two or more people can compete to see who is the happiest, i.e. maintaining the highest happiness level for the longest time.

(iii) Drug Delivery Based on Brainwave Feedback

Embodiments of the invention can be part of a feedback system that receives brainwave information from the patient and determines whether the current brainwave state is below the desired ‘healthy threshold such as negative emotions, anxiety or pain. If the emotional state becomes negative, the system will trigger an implanted or external drug delivery device to release antidepressant or endorphins into the patients’ body, such that negative emotions can be relieved and a more positive emotional state can be attained.

(iv) Social Robots

(iv-i) Interaction With and Training

The utility of embodiments of the present invention can translate to the realm of social robots, where the detection of human brainwaves will be transmitted wireless to the robots. The robot can then sense the brainwave state of the user and provide suitable responses to improve the user's mood. This approach can help to create empathic robots which may be important in counseling depressed users. Example embodiments can also be used to refine and improve on certain activities performed by the robots until the human is satisfied with its performance. The emotional scale of both the human and the robot can be displayed and tracked before, during and after the activity to be performed. For example, the human feels disappointed or sad when the robot is unable to lift a 5 kg weight. The robot detects the undesired brainwave and tries harder to achieve better results. Upon repeated success, the robot succeeds and it exhibits facial happiness. Both the brainwaves of the human and the emotional scale of the robot can be displayed based on the task-specific activity. This could serve as a humanized-robotic model for various purposes including, but not limited to, parenting to demonstrate that growth and development is based on intrinsic motivation.

(iv-ii) Alternative-Communicator Using Robots as a Representative

For people who have difficulties in facial and verbal expression (e.g. comatose or multiple sclerosis patients), a robot representative can be used by the individual in replacement for his/her disability to expressing brainwaves through facial, verbal and physical methods, in example embodiments. This can become a communication tool to facilitate communication between people who are unable to talk or express themselves, where the brainwaves of the individual could be channeled wirelessly to the robot, who will express the respective brainwaves displayed with varying brainwave levels. The robot can then act as a communicator in person or on webcam for personal/distant communication through virtual video.

(v) Remote Control

The EEG device in example embodiments can be combined with positional sensors, such as the inertial measurement unit (IMU), to permit direction-targeted remote control of objects in a 3D environment. For example, an IMU-EEG integrated system can collect EEG data (e.g. attention) coupled with head orientation, such that an increased mental focus in a certain direction can be utilized to remotely perform certain tasks. For instance, an attachable actuator with embedded transceiver can be placed on a rocker switch, so that when the user gives a mental EEG command facing the actuator, the switch will be activated by the actuator and therefore lights up the bulb. Another example would be attachable wheels (with embedded transceiver) which can be placed under a toy car, so that when the user gives a mental EEG command facing the wheels, the toy car will move towards the user. Another possible use would be an attachable vibrating motor (with embedded transceiver) which can be placed onto a drinking cup, so that when the user gives a mental EEG command facing the vibrating motor, the cup vibrates. Potential users include general consumers (adults and children), and also patients with physical disability such as stroke-related paralysis, Parkinson's disease, muscle dystrophy, multiple sclerosis etc. In addition, the system can be modified for use in remote control of assistive devices, for example, a user with paraplegia dons the EEG device and is able to send EEG command, coupled with his/her head orientation, to control the direction of motion of the wheelchair.

In preferred embodiments, a system is provided that provides active intervention, upon detection of specific brainwave patterns. The active intervention can be by means of, but is not limited to, a mechanical stimulus/movement such as vibration or a sensory stimulus (temperature, prick), visual, auditory, olfactory stimulus.

The device in such embodiments can have additional attachments or embedded features such as but not limited to temperature-sensitive pads that can cause a change in temperature e.g. to decrease body temperature due to rising heat from anxiety via activation of the temperature-sensitive pads or activation of motor(s) for a mechanical stimulus.

The location of the stimulus can include, but is not limited to, the neck, temples, shoulder, back, back of ear, feet areas either individually or in combination.

The sensory stimulus may serve one or multiple interventions such as, but not limited to, massaging the temples to calm the user, poking in order to wake the user, regulating the body temperature and emitting lavender scent to provide mental relief.

The active intervention means can exist as part of the brainwave-sensing device in its entirety or can be integrated for use with independent therapeutic systems such as, but not limited to, a massage chair/hugging jacket etc.

Depending on the level of e.g. anxiety, the active system in such embodiment can be programmed to be activated accordingly, with relevant magnitude or frequency, to the preferred mode of intervention such as mechanical stimulus, the rhythm, intensity, massage duration.

FIGS. 35A)-C) are schematic drawings illustrating examples of embodiments for implementation of the active interventions (mechanical, temperature, olfactory stimulus) that exist as part of the brainwave-sensing device 3500, 3502 3504 in entirety. More specifically, device 3500 has integrated vibration motors 3506 in addition to electrodes 3508, device 3502 has integrated temperature regulating pads 3509 in addition to electrodes 3510, and device 3504 has integrated olfactory emitting pads 3512 in addition to electrodes 3514. One or more types of the active means can be provided in one device in different embodiments.

Examples of embodiments in which the brainwave-sensing device exists independently of other systems such as a massage chair/vest that can be integrated for use together are illustrated in FIGS. 36 and 37. In FIG. 36, when a threshold level of mental state(s) is reached as measured by the brainwave-sensing device 3600, the intervention will be activated through communication between the device 3600 and a control unit (not shown) of the massage chair 3602, triggering motor(s) e.g. 3604 activation in the massage chair 3602, for example to reduce anxiety, or more generally to equilibrate the mental state back to healthy levels for optimal performance. Similarly in FIG. 37, when a threshold level of mental state(s) is reached as measured by the brainwave-sensing device 3700, the intervention will be activated through communication between the device 3700 and a control unit (not shown) of the massage vest 3702, triggering motor(s) e.g. 3704 activation in the massage vest 3702, for example to reduce anxiety, or more generally to equilibrate the mental state back to healthy levels for optimal performance.

In example embodiments such as, but not limited to, the example embodiments described with reference to FIGS. 36 and 37, the device can also be configured to receive a user input, e.g. so as to direct where an intervention should be located, such as which of a plurality of massaging motors should be activated upon detecting a certain brainwave activity/state.

FIG. 38 illustrates detection of a specific level of a mental state, e.g. high anxiety, represented by a peak 3800 in the brainwave activity measurement (compare plot 3802), activating vibration motors 3804 on the for example, wearable EEG device 3806 to provide massaging motion, and thereby normalizing the mental state, as represented by the “flat” brainwave activity in subsequent measurements (compare plot 3808). It will be appreciated that the principles illustrated in FIG. 38 are not limited to activation of vibration motors, but can be extended to other active components. the active components may be provided on the EEG device or may be external to the EEG device.

Depending on the level of the mental state detected, a corresponding set of motor vibrations, comprising of relevant features such as, but not limited to, magnitude or frequency, can be activated in example embodiments, as illustrated in FIGS. 39A) and B) for, as an example, high and low anxiety levels respectively.

### Example Embodiments May Have One or More of the Following Characteristics

(1) Providing a brainwave-sensing device, including wearables such as headwear, forehead patch, accessories, but also hand-held equipment in a variety of different form factors such as, but not limited to, a pistol-like design

(2) Allows detection of mental states such as emotions (happiness, anger, sadness, fear, excitement), pain, anxiety, sleep, mental fatigue, comfort and pleasure, upon placement of electrodes against the user's head.

(3) The form factor of the device can be modified and/or customized to suit individual use-cases, applications or personal preferences. This may also allow comfortable fitting of the headband on heads of all sizes as well as to allow compact storage of the device when it is not in use.

(4) The device can have a modular design. For example, the wearable device in its entirety can comprise of multiple modules which can be disassembled, then refitted or combined with another module for different sizes, use-cases, aesthetics (shape/design) etc.

(5) The device can have a telescopic design. Telescopic mechanisms allow the sliding of sections which can fit into each other. The sections can slide so as to allow fitting on heads of all sizes and allow compact storage when not in use.

(6) The device can have an extendibility/attachment feature which allows additional electrodes/components such as, but not limited to, external sensors (heart rate monitor) to be added to the device. This allows monitoring of other useful information such as heart rate, temperature etc. for the individual applications of interest to supplement the brainwave information obtained.

(7) The device can have a collapsible structure. This allows the device to be folded into a smaller size without compromising structural integrity.

(8) Flexible placement of electrodes along the device. The device can have multiple electrodes which can be configured in multiple ways to allow for different use cases. Electrodes can be adjusted or removed from the original position and repositioned onto desired areas; additional electrodes and/or sensors can be added to the device.

Advantageously, the reference electrodes of the device are not restricted to placement on bony areas of the head and can be moved to more convenient locations. This allows user to place the electrodes on the appropriate locations to suit the different use-case or for the purpose of greater comfort without compromising accuracy of the system.

The addition of electrodes to the device can provide users with a broader range of brainwave information at different parts of the brain; or the addition of sensors such as heart rate or temperature sensors for measurement of other physiological signals. This expands the scope of use of the device in its ability to read brainwave data whilst correlating it with other vital signs.

(9) The device can have a design that allows electrodes/sensors to be attached in several different ways. For example, the device can have holes/grooves located on necessary locations to fit electrodes into the device; electrodes can be attached with conductive fasteners such as Velcro strips; electrodes can be magnetized with the device with opposite polarities. This allows easy relocation of electrodes or addition of electrodes/sensors to suit the different use-cases.

(10) The conductive electrodes used in example embodiments of the device can be dry electrodes. Dry electrodes can provide greater convenience and can be used directly without requiring application of gels onto the electrodes in order to provide a conductive medium for brainwave sensing. These dry electrodes can advantageously collect data independent of hair length and thickness and compensate for varying scalp conductance.

(11) An algorithm to identify the user's mental state and its associated levels in example embodiments can use a supervised approach that requires the user to exhibit a specific mental state repeatedly so that the detected brainwave profile can be tagged to the desired mental state. With increased repetition or training data, the computer action becomes tagged to the desired mental state. By way of non-limiting example only, reference is made to the supervised approach described in Empirical Evaluation of the Emotiv EPOC BCI Headset for the Detection of Mental Actions (http://pro.sagepub.com/content/56/1/193.abstract).

(12) The algorithm can allow unsupervised detection of mental states such as, but not limited to, anxiety levels., i.e. without requiring human input. This approach uses a predetermined relationship between the specific mental state and the brainwave profile, based on previously collected test data from a subject population. The algorithm can allow mental state identification, which can be especially useful for users who are unable to provide user input, e.g. vegetative patients, patients with mental disorders, elderly with dementia. The unsupervised approach allows healthcare workers to monitor the mental states, e.g. anxiety levels of a patient/trainee, which can aid in early intervention whenever necessary. Unsupervised algorithms rely on a pre-determined pattern (unsupervised), for example, but not limited to, attention detection, e.g. low amplitudes of alpha waves at the frontal cortex are scientifically reported to be associated with strong attention levels. Hence, if the EEG device detects low alpha amplitudes at the frontal cortex of the wearer, a corresponding high attention level can be notified by the device.

(13) The algorithm can also allow therapeutic interventions to be implemented such as playing calm music when it detects high anxiety as the users' mental state, etc. The algorithm can also be able to provide feedback to the user to help in reducing e.g. anxiety levels when high levels are detected.

(14) The algorithm used in example embodiments can preferably identify the mental state of the user and its associated levels, particularly patients and athletes, in an automated manner. The supervised approach allows for an adaptive user-specific mental state identification, based on regular user inputs. Over time, a significant amount of mental state data can be collected from a large user base, leading to a more robust identification algorithm.

(15) Example embodiments can provide a mobile app platform whereby the raw and/or processed brainwave (and/or other physiological) information can be transmitted to and displayed on the mobile phone for user to utilize in a convenient and meaningful manner.

Users are able to monitor and track their mental states at their convenience through a mobile app, and take the necessary preventive or interventive steps as required.

(16) The flexible placement of electrodes in example embodiments can allow easy relocation of electrodes along the device to suit the different use-cases. Hence one system can be suited to monitor multiple applications. Well-embedded less visible/non-visible electrodes may be better received by the public as a daily ‘cool’ wearable tool.

In one embodiment, a system for measuring and processing brainwave data of a user is provided. The system comprises one or more electrodes for measuring the brainwave data of the user, and a processing unit coupled to the electrodes and configured to process the brainwave data for determining a current mental state of the user; and generate, based on the current mental state of the user, a control signal for instructing activation of a means for manipulating the current mental state of the user.

The processing unit may further be configured to process the brainwave data for monitoring a change in the mental state of the user, and to modify the first control signal based on the change.

The processing unit may further be configured to modify the control signal to change an amplitude and/or a frequency of the means for manipulating the current mental state of the user.

The system may further comprising an interface for communicating the control signal to a device external to the system. The external device may comprise a massage apparatus. The massage apparatus may comprise one or more of a group consisting of a massage chair, a massage cushion, a wearable massage appliance such as a massage vest, a water-based stimulation device such as a Jacuzzi, and an electronic pulse/electrical stimulation device. The external device may comprise one or more of a group consisting of a mechanical stimulation device, a temperature regulating device, a display device, an audio device and an olfactory emitting device.

The system may further comprise a manipulation component configured to receive the control signal. The system may comprises an instrument for carrying the one or more electrodes, and the manipulation component is disposed on the instrument. The instrument may comprise a portable unit such as a wearable unit or a hand-held unit. The manipulation component may comprise one or more of a group consisting of a mechanical stimulation element, a temperature regulating element, a display element, an audio element and an olfactory emitting element.

The manipulation of the current mental state may be for providing mental relief such as for reducing anxiety or promoting happiness.

The system 4000 may comprise an instrument for carrying the one or more electrodes. The instrument may comprise a portable unit such as a wearable unit or a hand-held unit.

The control signal may be configured to instruct presentation of one or more therapeutic solutions for selection by the user.

The control signal may be configured to instruct presentation of advertisements to the user.

The control signal may be configured to instruct notification of the current mental state of the user to a third party. The system may be configured to notify the third party through one or more of a group consisting of a video call, a voice call, a text message and a social networking platform. The system may be configured to notify the third party in a manner suitable for tracking the mental state of the user.

The control signal may be configured for one or more of a group consisting of controlling a vehicle driven by the user, activating a therapeutic measure, activating an alarm and activating a drug delivery.

The control signal may be configured to instruct a rating of a user experience associated with the current mental state. The user experience may comprise one or more of a group consisting of a shopping mall, a restaurant, a menu, a dish, an entertainment and a customer service experience.

The control signal may be configured to instruct an input into a computer game.

The control signal may be configured to instruct a robotic or remotely controlled device.

The processing unit may further be configured to generate the control signal based on a user input signal.

FIG. 40 shows a flow chart 4000 illustrating a method for measuring and processing brainwave data of a user, according to an example embodiment. At step 4002 one or more electrodes for measuring the brainwave data of the user are provided. At step 4004, the brainwave data is processed for determining a current mental state of the user. At step 4006, a control signal for instructing activation of a means for manipulating the current mental state of the user is generated based on the current mental state of the user.

The method may further comprise processing the brainwave data for monitoring a change in the mental state of the user, and modifying the first control signal based on the change.

The method may comprise modifying the control signal to change an amplitude and/or a frequency of the means for manipulating the current mental state of the user.

The method may further comprise communicating the control signal to an external device. The external device may comprise a massage apparatus. The massage apparatus may comprise one or more of a group consisting of a massage chair, a massage cushion, a wearable massage appliance such as a massage vest, a water-based stimulation device such as a Jacuzzi, and an electronic pulse/electrical stimulation device. The external device may comprise one or more of a group consisting of a mechanical stimulation device, a temperature regulating device, a display device, an audio device and an olfactory emitting device.

The method may further comprise receiving the control signal at a manipulation component. The method may comprise disposing the manipulation component on an instrument for carrying the one or more electrodes. The instrument may comprise a portable unit such as a wearable unit or a hand-held unit. The manipulation component may comprise one or more of a group consisting of a mechanical stimulation element, a temperature regulating element, a display element, an audio element and an olfactory emitting element.

The manipulation of the current mental state may be for providing mental relief such as for reducing anxiety or promoting happiness.

The method may comprise providing an instrument for carrying the one or more electrodes. The instrument may comprise a portable unit such as a wearable unit or a hand-held unit.

The method may comprise instructing, using the control signal, presentation of one or more therapeutic solutions for selection by the user.

The method may comprise instructing, using the control signal, presentation of advertisements to the user.

The method may comprise instructing, using the control signal, notification of the current mental state of the user to a third party. The method may comprise notifying the third party through one or more of a group consisting of a video call, a voice call, a text message and a social networking platform. The method may comprise notifying the third party in a manner suitable for tracking the mental state of the user.

The method may comprise one or more of a group consisting of controlling a vehicle driven by the user, activating a therapeutic measure, activating an alarm and activating a drug delivery, using the control signal.

The method may comprise instructing, using the control signal, a rating of a user experience associated with the current mental state. The user experience may comprise one or more of a group consisting of a shopping mall, a restaurant, a menu, a dish, an entertainment and a customer service experience.

The method may comprise instructing, using the control signal, an input into a computer game.

The method may comprise instructing, using the control signal, a robotic or remotely controlled device.

The method may comprise generating the control signal based on a user input signal.

In one embodiment, a device for measuring brainwave data of a user is provided. The device comprises a portable instrument; and one or more electrodes disposed on, or for disposal on the portable instrument; wherein the portable instrument and/or the electrodes are configured for providing an adjustable configuration of the one or more electrodes for measurement of the brainwave data.

The portable instrument may comprise a wearable unit and/or a hand-held unit.

The adjustable configuration for measurement of the brainwave data may comprise one or more of frontal, occipital and temporal.

The portable instrument and/or the one or more electrodes may be configured to enable adjustment of a relative position of the electrodes to each other on the portable instrument.

The portable instrument and/or the one or more electrodes may be configured to enable adjustment of the number of the electrodes disposed on the portable instrument.

The portable instrument comprises one or more channels and/or holes may be configured for releasably disposing the electrodes on the portable instrument. The one or more channels and/or holes may be configured for other attachment elements such as sensors, adaptors associated with the sensors, extension ports associated with the sensors, or retractable instruments associated with the sensors. The electrodes may be lockable at different locations along the channels.

The portable instrument may comprise one or more conductive linings for releasably attaching the electrodes.

The conductive linings may comprise one or more of a group consisting of magnetic fasteners, mechanical fasteners, and adhesive fasteners.

The portable instrument may comprise one or more of a group consisting of a collapsible structure, a telescopic structure, a flexible bendable structure, a foldable structure and a modular structure.

FIG. 41 shows a flow chart 4100 illustrating a method for measuring brainwave data of a user, according to an example embodiment. At step 4102, a portable instrument is provided. At step 4104, one or more electrodes disposed on, or for disposal on the portable instrument are provided. At step 4106, an adjustable configuration of the one or more electrodes for measurement of the brainwave data is provided.

The portable instrument may comprise a wearable unit and/or a hand-held unit.

The adjustable configuration for measurement of the brainwave data may comprise one or more of frontal, occipital and temporal.

The method may comprise adjustment of a relative position of the electrodes to each other on the portable instrument.

The method may comprise adjustment of the number of the electrodes disposed on the portable instrument.

The method may comprise releasably disposing the electrodes on the portable instrument. The method may comprise attachment of elements such as sensors, adaptors associated with the sensors, extension ports associated with the sensors, or retractable instruments associated with the sensors on the instrument. The method may comprise locking the electrodes at different locations along the channels.

The method may comprise using one or more conductive linings for releasably attaching the electrodes on the instrument.

The conductive linings may comprise one or more of a group consisting of magnetic fasteners, mechanical fasteners, and adhesive fasteners.

The portable instrument may comprise one or more of a group consisting of a collapsible structure, a telescopic structure, a flexible bendable structure, a foldable structure and a modular structure.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features, in particular any combination of features in the patent claims, even if the feature or combination of features is not explicitly specified in the patent claims or the present embodiments.

For example, embodiments of the present invention can be adapted to detect brainwaves localized at all parts of the brain, including but not limited to emotions, memory, motor skills, hearing, vision and speech, during both static and dynamic tasks using the methodology and/or the various applications as described.

Also, a modification of the preferred embodiments can be the potential integration of the brainwave detection device and brainwave identification software with consumer electronics such as computers, tablets, smart phones, cameras (both handheld or computer cameras). Such integration can offer an approach for mass adoption of the brainwave detection technology, while at the same time providing a convenient tool for identification of user brainwaves for the aforementioned applications, but not limited to those applications.

Furthermore, example embodiments of the present invention can be applicable to all parts of the brain, such as, but not limited to, emotions, memory, motor skills, hearing, vision and speech functions.

1. A system for measuring and processing brainwave data of a user, the system comprising:
one or more electrodes for measuring the brainwave data of the user, and
a processing unit coupled to the electrodes and configured to:
process the brainwave data for determining a current mental state of the user; and
generate, based on the current mental state of the user, a control signal for instructing activation of a means for manipulating the current mental state of the user.

2. The system as claimed in claim 1, wherein the processing unit is further configured to process the brainwave data for monitoring a change in the mental state of the user, and to modify the first control signal based on the change.

3. The system as claimed in claim 1, wherein the processing unit is configured to modify the control signal to change an amplitude and/or a frequency of the means for manipulating the current mental state of the user.

4-8. (canceled)

9. The system as claimed in claim 1, wherein the system comprises a portable unit such as a wearable unit or a hand-held unit an for carrying the one or more electrodes, and a manipulation component configured to receive the control signal, the manipulation component being disposed on the instrument.

10-14. (canceled)

15. The system as claimed in claim 1, wherein the control signal is configured to instruct one or more of a group consisting of presentation of one or more therapeutic solutions for selection by the user, presentation of advertisements to the user, and notification of the current mental state of the user to a third party.

16-19. (canceled)

20. The system as claimed in claim 1, wherein the control signal is configured for one or more of a group consisting of controlling a vehicle driven by the user, activating a therapeutic measure, activating an alarm and activating a drug delivery, instructing a rating of a user experience associated with the current mental state, instructing an input into a computer game, instructing a robotic or remotely controlled device.

21-24. (canceled)

25. The system as claimed in claim 1, wherein the processing unit is further configured to generate the control signal based on a user input signal.

26. A method for measuring and processing brainwave data of a user, the method comprising:
providing one or more electrodes for measuring the brainwave data of the user,
processing the brainwave data for determining a current mental state of the user; and
generating, based on the current mental state of the user, a control signal for instructing activation of a means for manipulating the current mental state of the user.

27. The method as claimed in claim 26, further comprising processing the brainwave data for monitoring a change in the mental state of the user, and modifying the first control signal based on the change.

28. The method as claimed in claim 26, comprising modifying the control signal to change an amplitude and/or a frequency of the means for manipulating the current mental state of the user.

29-33. (canceled)

34. The method as claimed in claim 33, wherein the method comprises disposing a manipulation component for receiving the control signal on a portable unit such as a wearable unit or a hand-held unit an instrument for carrying the one or more electrodes.

35-39. (canceled)

40. The method as claimed in claim 16, comprising instructing, using the control signal, one or more of a group consisting of presentation of one or more therapeutic solutions for selection by the user, presentation of advertisements to the user, and notification of the current mental state of the user to a third party.

41-44. (canceled)

45. The method as claimed in claim 26, comprising using the control signal for one or more of a group consisting of controlling a vehicle driven by the user, activating a therapeutic measure, activating an alarm, instructing a rating of a user experience associated with the current mental state, instructing an input into a computer game, instructing a robotic or remotely controlled device, and activating a drug delivery.

46-49. (canceled)

50. The method as claimed in claim 26, comprising generating the control signal based on a user input signal.

51. A device for measuring brainwave data of a user, the device comprising:
a portable instrument; and
one or more electrodes disposed on, or for disposal on the portable instrument;
wherein the portable instrument and/or the electrodes are configured for providing an adjustable configuration of the one or more electrodes for measurement of the brainwave data.

52. The device as claimed in claim 51, wherein the portable instrument comprises a wearable unit and/or a hand-held unit.

53. The device as claimed in claim 51, wherein the adjustable configuration for measurement of the brainwave data comprises one or more of frontal, occipital and temporal.

54. The device as claimed in claim 51, wherein the portable instrument and/or the one or more electrodes are configured to enable one or more of a group consisting of adjustment of a relative position of the electrodes to each other on the portable instrument and adjustment of the number of the electrodes disposed on the portable instrument.

55-60. (canceled)

61. The device as claimed in claim 51, wherein the portable instrument comprises one or more of a group consisting of a collapsible structure, a telescopic structure, a flexible bendable structure, a foldable structure and a modular structure.

62. A method for measuring brainwave data of a user, the device comprising:
providing a portable instrument;
providing one or more electrodes disposed on, or for disposal on the portable instrument; and
providing an adjustable configuration of the one or more electrodes for measurement of the brainwave data.

63. The method as claimed in claim 62, wherein the portable instrument comprises a wearable unit and/or a hand-held unit.

64. The method as claimed in claim 62, wherein the adjustable configuration for measurement of the brainwave data comprises one or more of frontal, occipital and temporal.

65. The method as claimed in claim 62, comprising one or more of a group consisting of adjustment of a relative position of the electrodes to each other on the portable instrument and adjustment of the number of the electrodes disposed on the portable instrument.

66-71. (canceled)

72. The method as claimed in claim 62, wherein the portable instrument comprises one or more of a group consisting of a collapsible structure, a telescopic structure, a flexible bendable structure, a foldable structure and a modular structure.
